(19) United States
(12) Patent Application Publication
(10) Pub. No.: US 2026/0341396 A1
(43) Pub. Date: Oct. 29, 2026

(54) Recognition-Quantum Bio-Folding Engine Methods and Hardware for Parameter-Free Protein Folding and RNA Kinetics Using Integer Energy Ladders and Quantum Tunnelling Cross-Reference to Related Applications

(71) Applicant: Jonathan Washburn, Austin, TX (US)

(72) Inventor: Jonathan Washburn, Austin, TX (US)

(21) Appl. No.: 19/652,167

(22) Filed: Apr. 20, 2026

Related U.S. Application Data
(60) Provisional application No. 63/794,532, filed on Apr. 25, 2025.

(51) Int. Cl. G16B 5/00; G16B 15/20

(52) U.S. Cl. CPC G16B 5/00; G16B 15/20

(57) ABSTRACT

A biomolecular simulation engine computes protein folding pathways, predicted structures, and RNA polymerase kinetics using a fixed cohesion quantum and integer energy ladders without fitted force-field energy parameters. Discrete biomolecular states are assigned integer ladder indices, and state energies are computed as integer multiples of the fixed cohesion quantum. A discrete state graph is constructed from permitted state transitions, and nonnegative transition costs derived from ladder energies and, in some embodiments, quantum-tunneling transition rules are used to determine a pathway through the graph. For protein folding, the pathway is used to reconstruct a predicted three-dimensional structure. For RNA kinetics, position-dependent ladder indices are used to compute translocation rates, dwell times, and pause-density profiles. Embodiments include co-transcriptional folding, ensemble averaging, GPU and FPGA acceleration, validation workflows, formal verification, drug-ranking applications, and cloud-based simulation services.

### CROSS-REFERENCE TO RELATED APPLICATIONS

This application claims the benefit of U.S. Provisional Patent Application No. 63/794,532, filed on Apr. 25, 2025, the entire disclosure of which is incorporated herein by reference to the extent permitted and to the extent not inconsistent with the present disclosure.

### BACKGROUND OF THE INVENTION

The present disclosure relates generally to biomolecular simulation, structural biology, computational biophysics, enzyme kinetics, and machine-implemented prediction of biomolecular behavior. More particularly, the present disclosure relates to systems and methods for predicting protein folding pathways, folded conformations, RNA folding behavior, transcription-coupled structure formation, and enzyme translocation kinetics using a fixed cohesion quantum, integer energy ladders, graph-based state transitions, and quantum-tunneling transition rules, while reducing or eliminating reliance on fitted molecular-mechanics force-field energy parameters.

A longstanding technical problem in biomolecular modeling is that many widely used approaches depend on large families of empirically tuned parameters that are selected, calibrated, or reweighted against experimental datasets, quantum-chemistry calculations, or both. In conventional molecular dynamics and related simulation frameworks, the state of a biomolecule is evolved using energy functions that typically decompose into bonded and non-bonded terms, such as bond stretch terms, angle bend terms, torsional terms, electrostatic terms, and van der Waals terms. Those terms are often further stratified by atom type, bond type, residue environment, charge model, solvent assumptions, and correction terms. As a result, practical implementations frequently depend on extensive parameter tables, fitted coefficients, and modeling conventions that differ across force-field families and software environments. This creates a technical reproducibility problem because the same protein, nucleic acid segment, or enzyme system may produce materially different predicted pathways, state occupancies, or folded structures depending on which force field, solvent model, minimization policy, cutoff policy, protonation assumption, or restraint configuration is selected.

That dependence on parameterization is not merely a theoretical inconvenience. It affects computational control flow, convergence behavior, reproducibility of results, transportability across compute environments, and auditability during validation and review. When a structure prediction or kinetic prediction depends on hundreds or thousands of interacting coefficients, it becomes difficult to determine whether an observed output reflects the underlying molecule, the particular model family, or a hidden calibration choice. This matters operationally because developers, researchers, and downstream systems often need a deterministic and explainable basis for comparing multiple molecules, ranking candidate compounds, reproducing folding trajectories, or validating kinetic outputs across benchmark datasets and hardware platforms. A model that changes materially when parameters are re-tuned or when one fitted force-field family is substituted for another creates technical uncertainty in deployment, weakens traceability, and complicates examiner review of whether the disclosed invention actually provides a distinct and reproducible computational mechanism.

Conventional molecular dynamics approaches also impose substantial computational cost because they generally evolve biomolecular systems through large numbers of time steps while repeatedly evaluating high-dimensional interaction terms. Even when such methods are effective for some classes of systems, they can require substantial memory bandwidth, extensive floating-point computation, and long runtimes, particularly for larger proteins, multistate conformational searches, or nucleic acid systems that are evaluated over long simulated trajectories. In practice, this can make exhaustive or repeated pathway exploration difficult, especially where many candidate molecules, many sequence variants, or many operating conditions must be evaluated. In a production, cloud, or accelerator-based environment, such cost directly affects throughput, power consumption, scheduling, queue depth, and the feasibility of integrating folding and kinetic calculations into a unified pipeline.

Another technical shortcoming of many classical biomolecular simulation methods is that they typically represent the energy landscape as a largely continuous potential surface and rely on continuous-state evolution under classical dynamics assumptions. Such treatments can be useful in many contexts, but they do not natively provide a discrete, machine-actionable state-transition model in which conformational changes and kinetic events are represented as transitions among integer-indexed energy states governed by explicit transition rules. This matters because many practical computational workflows benefit from discrete state representations. Discrete states enable bounded graph construction, objective transition costs, deterministic path search, formal verification of cost properties, sparse accelerator implementations, and compact storage of intermediate states. When a modeling framework lacks such a discrete architecture, it is harder to produce minimum-cost pathway outputs, harder to apply shortest-path logic, and harder to guarantee nonnegative and additive transition metrics suitable for deterministic graph solvers.

A further technical limitation arises in relation to quantum effects and tunneling-like behavior in biomolecular systems. A variety of biological mechanisms have been associated in the literature with state changes that are not well characterized as purely classical smooth descent on a continuous force surface. Examples discussed in the field include proton transfer, hydrogen-bond rearrangement, base-pair opening and closing phenomena, and polymerase movement behavior reflected in pause-density patterns. Purely classical force-field evolution does not inherently provide an explicit tunneling transition rule tied to a compact state representation. At the other end of the spectrum, ab initio quantum chemistry can in principle model quantum behavior more directly, but it is computationally expensive and commonly scales poorly with increasing system size, making it impractical for many large biomolecules, whole-sequence scans, polymerase-scale kinetics, or repeated benchmarking across many inputs. This leaves a technical gap between computational tractability and explicit treatment of discrete transition behavior associated with tunneling-type events.

Protein folding presents a particularly acute version of this gap. Existing approaches often either rely on expensive trajectory simulation, machine-learned inference systems trained on large structure corpora, or heuristic scoring functions that remain dependent on calibration, templates, or learned priors. Some systems predict a final structure without providing an explicit folding pathway in a deterministic state-transition sense. Others provide trajectories, but those trajectories may be expensive to compute, difficult to interpret, and heavily dependent on the selected potential function and simulation policy. For many scientific and engineering uses, however, the pathway is itself a technically important output. A pathway can determine intermediate states, trapped states, transition bottlenecks, kinetic accessibility, state ordering, and controllable intervention points. Where a computational system does not yield an explicit minimum-cost or otherwise objectively ranked pathway through a discrete conformation graph, the user may receive only an end-state hypothesis without the control logic needed to analyze how the system got there, why a given state was selected, or which state transitions dominate the result.

RNA and enzyme kinetics present related shortcomings. In polymerase systems, useful outputs often include not merely a single rate constant but a position-dependent kinetic profile, dwell-time distribution, pause-density profile, or timing signal that can be coupled to downstream structure formation. Existing methods may rely on fitted sequence models, hidden-state models, empirical regression layers, or simulation approaches that are specialized to one enzyme family, one assay type, or one calibration regime. That can make it difficult to maintain a unified architecture across protein folding, RNA kinetics, and co-transcriptional folding. It can also make it difficult to preserve a single underlying state semantics across different biomolecular tasks. When folding and kinetics are computed by unrelated engines using unrelated energy models, the timing outputs from one engine are not naturally commensurate with the state transitions of the other. That architectural separation can prevent or weaken technically coherent simulation of nascent transcript folding during transcription, because the available folding window at each transcript position depends on polymerase timing, while the accessible structure states depend on the folding state representation.

There is therefore a technical need for a unified computational architecture in which biomolecular states can be represented using a common quantized energy logic, pathway costs can be computed using objective and nonnegative rules, kinetics can be derived from the same underlying ladder representation, and both folding and translocation behavior can be executed in a deterministic, reproducible, accelerator-friendly manner. Such an architecture should preferably reduce or eliminate the need for large fitted force-field energy tables, provide explicit support for discrete transition behavior including tunneling-type transitions, support graph-based solution procedures such as shortest-path search, and produce outputs that are not limited to final structures but also include pathways, rates, dwell times, pause densities, intermediate states, and machine-actionable ranking values.

The technical need is not limited to accuracy in the abstract. Examiner review and real-world deployment both benefit from disclosures that define objective computational rules rather than merely reciting result-oriented aspirations. A useful biomolecular engine should disclose what states are formed, how indices are assigned, how costs are computed, how transitions are permitted or disallowed, how outputs are reconstructed, how ensemble properties are computed, how hardware acceleration is applied, and how benchmark or validation outputs are generated. Without that level of implementation detail, a purported folding or kinetics engine may amount only to an aspirational statement that a desired biological prediction is produced. Such a disclosure can be vulnerable to criticism that it lacks concrete mechanism, lacks objective decision boundaries, or merely combines familiar components without demonstrating a specific architecture that causes a specific technical result.

The present disclosure addresses these and other shortcomings by providing a parameter-reduced or parameter-free biomolecular simulation framework in which a fixed cohesion quantum is used as an energy step, biomolecular state energies are represented as integer multiples of that fixed step, transitions are governed by explicit ladder and tunneling rules, pathway search is performed over discrete graph structures using nonnegative costs, and kinetics can be computed using ladder-index-based rate laws under the same architectural framework. In this way, the disclosed subject matter is directed to a concrete technical solution to the technical problems of force-field dependence, poor reproducibility across parameterizations, limited pathway determinism, poor support for tunneling-type discrete transitions, fragmented folding-versus-kinetics architectures, and high computational burden associated with large-scale continuous-state simulation.

### SUMMARY OF THE INVENTION

Disclosed herein are systems, methods, devices, accelerators, services, and machine-readable implementations for biomolecular simulation using a fixed cohesion quantum, integer energy ladders, and quantum-tunneling transition rules to compute folding pathways, folded structures, kinetic profiles, and related technical outputs without relying on fitted molecular-mechanics force-field energy parameter sets. In various embodiments, the disclosed subject matter provides a deterministic, machine-actionable architecture in which biomolecular state energies are constrained to integer multiples of a fixed energy quantum, transitions among those states are represented using explicit graph and transition rules, and outputs are generated through ordered computational operations that can be executed in software, hardware, firmware, or combinations thereof.

In some embodiments, the disclosed architecture fixes a cohesion quantum in a Recognition-Physics native form and also provides an eV-calibrated form for numerical computation. In some embodiments, the fixed cohesion quantum is predetermined for a selected implementation ruleset before pathway solving begins and is not tuned on a per-sequence basis during a simulation run. In some embodiments, the fixed cohesion quantum is used as a universal energy step for multiple classes of biomolecular states, including protein backbone states, side-chain states, nucleotide states, enzyme conformational states, binding-site states, or combinations thereof. In some embodiments, the system assigns each allowed state a ladder index, such as a nonnegative integer, and computes a state energy as an integer multiple of the fixed cohesion quantum. In this manner, the system replaces large fitted energy parameter families with a compact integer-indexed state-energy representation that is reproducible, traceable, and suitable for deterministic downstream computation.

In some embodiments, the disclosed subject matter includes a ladder index assignment engine configured to receive sequence information, state definitions, conformation definitions, or other biomolecular inputs and to assign integer ladder indices according to objective rules. For protein folding, the objective rules can include mapping discretized backbone torsion regions, rotamer classes, side-chain states, secondary-structure-compatible state bins, residue-context classes, or combinations thereof to ladder indices. For nucleic-acid kinetics, the objective rules can include mapping nucleotide identity, base-step identity, local sequence context, polymerase-context state, or combinations thereof to ladder indices. In some embodiments, the mapping is static and predefined. In some embodiments, the mapping is selected from a finite rule library. In some embodiments, the mapping is species-specific, assay-specific, molecule-type-specific, or task-specific while still preserving the integer-ladder architecture and fixed cohesion quantum.

In some embodiments, the disclosed subject matter includes a graph-construction engine configured to construct a discrete state graph from the ladder-indexed biomolecular states. In protein embodiments, nodes can represent allowed torsion states, rotamer states, residue-state tuples, residue-window states, or multi-residue conformation states. Edges can represent permissible transitions between states according to adjacency rules, compatibility rules, sequence ordering rules, steric admissibility rules, folding-direction rules, or combinations thereof. In some embodiments, the graph is layered by residue position, sequence position, or conformation stage, which can facilitate deterministic shortest-path or dynamic-programming evaluation. In some embodiments, the graph is sparse because only transitions satisfying predefined compatibility criteria are instantiated. In some embodiments, graph generation is performed in streaming fashion, in tiled fashion, or in fully materialized form depending on memory and performance requirements.

In some embodiments, the disclosed subject matter includes a transition-cost engine configured to compute nonnegative state-transition costs from ladder energies, ladder-index differences, tunneling costs, or combinations thereof. In some embodiments, the transition-cost engine computes a destination-state cost, a differential cost, an accumulated path cost, or a hybrid cost. In some embodiments, a quantum-tunneling rule is applied to transitions between adjacent or otherwise permitted ladder levels. In some embodiments, a tunneling weight is computed as an exponential function of the fixed cohesion quantum and a characteristic frequency term. In some embodiments, an additive transition cost is computed as the negative logarithm of the tunneling weight. In some embodiments, a locking relation causes the tunneling-derived additive cost to reduce to a fixed constant per ladder step, thereby simplifying graph evaluation and accelerator implementation. In some embodiments, the disclosed architecture therefore provides an explicit technical mechanism for representing tunneling-type transition behavior in a discrete and machine-solvable state space rather than relying on an implicit or purely continuous treatment.

In some embodiments, the disclosed subject matter includes a path-solver engine configured to determine a pathway through the discrete state graph using a shortest-path solver, dynamic-programming solver, heuristic search solver, branch-and-bound solver, or other ordered optimization engine operating on objective transition costs. In some embodiments, the path-solver engine uses Dijkstra's algorithm on nonnegative edge costs. In some embodiments, the path-solver engine uses A* search with an admissible heuristic. In some embodiments, the path-solver engine uses a layered dynamic-programming traversal for acyclic or effectively acyclic graph organizations. The resulting pathway can define a most-probable folding sequence, a minimum-cost state progression, a ranked set of candidate pathways, or a pathway ensemble. This provides a technical effect not limited to a final folded output, because the architecture also yields explicit intermediate-state ordering, state-to-state transition logic, cost attribution, and pathway reproducibility.

In some embodiments, the disclosed subject matter includes a structure-reconstruction engine configured to convert a selected torsion-state pathway into a three-dimensional structural output. In some embodiments, the structure-reconstruction engine converts internal coordinates to Cartesian coordinates using bond lengths, bond angles, torsion angles, chirality constraints, closure rules, or combinations thereof. In some embodiments, the structure-reconstruction engine outputs a final predicted structure, one or more intermediate structures, torsion-angle sequences, state traces, confidence annotations, pathway-cost annotations, residue-wise state labels, or combinations thereof. In some embodiments, the disclosed subject matter thereby produces not only a structure hypothesis but also a machine-auditable record of how the structure was generated from the underlying ladder-index and graph-transition logic.

In some embodiments, the disclosed subject matter includes an ensemble-averaging engine configured to compute thermodynamic or statistical outputs from multiple states and/or multiple pathways using Boltzmann factors derived from integer ladder energies. In some embodiments, the ensemble-averaging engine computes partition-function approximations, pathway-weighted observables, state occupancies, residue-wise expectations, transition probabilities, or temperature-dependent outputs. In some embodiments, the partition-function computation is factorized approximately across residues, residue groups, state blocks, or graph layers. In some embodiments, the ensemble-averaging engine is optional, such that one implementation may return a single minimum-cost pathway while another implementation returns an ensemble-informed output under the same underlying ladder formalism.

In some embodiments, the disclosed subject matter includes an RNA kinetics engine configured to compute position-dependent enzyme translocation rates using ladder indices assigned to nucleotide positions or other sequence-defined units. In some embodiments, the RNA kinetics engine computes a translocation rate at each position using an exponential rate law based on the ladder index and the fixed cohesion quantum. In some embodiments, dwell times are derived as inverse rates. In some embodiments, normalized pause-density profiles, pause histograms, ranked pause sites, dwell maps, kinetic bottleneck labels, or combinations thereof are generated from the dwell times. In some embodiments, the same fixed cohesion quantum and integer-ladder formalism used for folding is also used for the kinetics engine, which provides a common state semantics and a technically unified architecture across different biomolecular tasks.

In some embodiments, the disclosed subject matter includes a co-transcriptional folding engine configured to couple a translocation-timing output from the RNA kinetics engine to a folding-time window used by an RNA folding engine, protein folding engine, or nascent-structure engine. In some embodiments, the coupling is implemented through a timing interface that exposes transcript position, dwell time, accumulated elapsed time, pause-state labels, or combinations thereof to a structure engine operating on the nascent molecule. In some embodiments, a longer dwell time at a given position enlarges a folding window for the then-available transcript portion, thereby modifying which ladder-indexed structure states are reachable before the next transcription increment occurs. This provides a concrete technical mechanism for unified co-transcriptional simulation rather than merely juxtaposing independent folding and kinetics modules.

In some embodiments, the disclosed subject matter includes validation and benchmarking engines configured to compare predicted structures against reference structures, compare predicted kinetic outputs against reference kinetic datasets, compute RMSD values, compare histograms, compute rank-order agreement values, generate benchmark reports, or store traceability artifacts linking outputs to input sequences, constant values, graph policies, and solver settings. In some embodiments, benchmark constants and formal proof artifacts are stored to support reproducibility and auditability. In some embodiments, this supports technical review, examiner review, regulatory review, scientific review, or internal engineering validation by enabling the user to trace a generated output back to the fixed cohesion quantum, ladder assignment rules, graph construction rules, and transition-cost logic used to produce that output.

In some embodiments, the disclosed subject matter includes a formal-verification subsystem configured to prove or check one or more mathematical properties of the disclosed engine, such as nonnegativity of ladder energies, monotonicity of ladder energies with increasing ladder index, positivity or boundedness of tunneling weights, nonnegativity of path costs, positivity of computed kinetic rates, or consistency of one module with another module under shared constants and rules. In some embodiments, the formal-verification subsystem is implemented using a theorem-proving environment and stores proof artifacts, build metadata, and traceability records. This provides a technical effect of increasing implementation rigor and machine-verifiable reproducibility of the disclosed computational framework.

In some embodiments, the disclosed subject matter includes hardware-accelerated embodiments. In some embodiments, GPU implementations parallelize ladder assignment, graph construction, cost computation, pathway solving, ensemble averaging, rate computation, or combinations thereof across residues, positions, graph layers, or batched molecules. In some embodiments, FPGA implementations instantiate ladder lookup tables, tunneling engines, shortest-path pipelines, kinetics pipelines, and buffering logic in configurable hardware. In some embodiments, the disclosed architecture is particularly suitable for accelerator execution because the state space is discrete, the transition logic is explicit, the cost rules are objective, and the ladder energies can be represented compactly. As a result, some embodiments provide improvements in throughput, memory efficiency, deterministic scheduling, and deployment scalability relative to approaches that repeatedly evaluate large continuous-state force-field expressions over long trajectories.

In some embodiments, the disclosed subject matter includes cloud and distributed embodiments. In some embodiments, a network service receives sequence submissions, instantiates one or more simulation jobs, dispatches those jobs to CPU, GPU, FPGA, or hybrid resources, and returns structures, pathways, kinetic profiles, rankings, or reports. In some embodiments, the service supports synchronous execution, asynchronous execution, batch processing, queue-based execution, cached execution, repeatable benchmark replay, or combinations thereof. In some embodiments, outputs include both human-readable reports and machine-actionable artifacts, such as state traces, pathway files, graph snapshots, ranked candidate lists, pause-site records, or structured API responses.

In some embodiments, the disclosed subject matter includes drug-ranking, enzyme-design, nanopore-signal, membrane-protein, protein-protein docking, RNA-secondary-structure, and hybrid quantum-classical embodiments. In some embodiments, the same integer-ladder architecture is used to compute binding-site energy differences, rank candidate compounds, identify mutations predicted to alter local ladder states, model membrane insertion states, evaluate interface states for docking, predict ionic current levels as a function of ladder state, or assign portions of the computation to different hardware domains depending on whether a transition is tunneling-dominated or otherwise computationally specialized. These embodiments provide fallback claim support and demonstrate that the disclosed architecture is not restricted to a single biological assay or a single output modality.

Accordingly, in one aspect, the invention provides a biomolecular simulation system comprising a memory storing instructions and one or more processors or accelerators configured to execute the instructions to receive biomolecular input data, assign integer ladder indices to biomolecular states using a fixed cohesion quantum, construct a discrete state graph, compute nonnegative transition costs including ladder-derived and optionally tunneling-derived costs, determine one or more pathways through the graph, and generate one or more outputs comprising a predicted structure, a folding pathway, a kinetic profile, or a pause-density output.

In another aspect, the invention provides a computer-implemented method for protein folding comprising receiving a protein sequence, assigning ladder indices to discretized torsion and optionally rotamer states, constructing a torsion-state graph, computing transition costs from integer ladder energies and optionally tunneling rules, solving for a minimum-cost pathway, and reconstructing a folded structure from the solved pathway.

In another aspect, the invention provides a computer-implemented method for enzyme or polymerase kinetics comprising receiving a nucleotide sequence, assigning ladder indices to sequence positions, computing position-dependent rates from the assigned ladder indices and the fixed cohesion quantum, computing dwell times and normalized pause-density values, and outputting a kinetic profile or pause histogram.

In another aspect, the invention provides a unified co-transcriptional simulation method comprising computing position-dependent translocation timing under the integer-ladder kinetics model, exposing that timing to a folding engine, and using the timing to constrain or select accessible folding states of a nascent transcript or other growing biomolecular structure.

In another aspect, the invention provides a hardware-accelerated biomolecular engine implemented on a GPU, FPGA, or hybrid platform and configured to execute one or more of ladder assignment, graph construction, transition-cost evaluation, pathway solving, ensemble averaging, and kinetics computation.

In another aspect, the invention provides non-transitory computer-readable media storing instructions that, when executed, cause one or more processors to perform any of the methods disclosed herein.

The disclosed subject matter therefore provides a technical solution to technical problems associated with force-field dependence, fragmented simulation architectures, continuous-state computational burden, limited pathway determinism, and lack of explicit tunneling-transition logic. By using a fixed cohesion quantum, integer energy ladders, explicit graph-state mechanics, and ordered transition rules, the disclosed architecture enables reproducible, deterministic, and accelerator-compatible generation of folding and kinetic outputs, while also supporting pathway traceability, formal verification, unified folding-and-kinetics modeling, and robust implementation alternatives suitable for prosecution, examination, and deployment.

### BRIEF DESCRIPTION OF THE DRAWINGS

FIG. 1 is a block diagram of an example Recognition-Quantum Bio-Folding system 100 including an input interface 102, sequence data 104, a preprocessing module 106, a ladder index assignment module 108, an integer ladder energy module 110, a torsion graph builder 112, a shortest-path solver 114, a Boltzmann averaging module 116, an RNA kinetics module 118, a co-transcriptional coupling module 120, an output generator 122, storage 124, an accelerator interface 126, a GPU accelerator 128, an FPGA accelerator 130, and a network interface 132.

FIG. 2 is a conceptual diagram of an integer energy ladder 200 showing an energy axis 202, an energy quantum step 204 corresponding to a fixed cohesion quantum, a ladder level index 206, ladder energy levels 208, one or more inter-level transition arrows 210, tunneling weight annotations 212, additive transition cost annotations 214, a locking relation annotation 216, a Boltzmann factor annotation 218, a temperature parameter annotation 220, an integer constraint annotation 222, and a state energy annotation 224.

FIG. 3 is a conceptual diagram of a torsion-state graph 300 for protein folding showing a residue i 302, a residue i+1 304, one or more torsion state nodes 306 for residue i, one or more torsion state nodes 308 for residue i+1, adjacency edges 310, edge cost labels 312, a Ramachandran region discretization 314, a ladder index assignment rule 316, a source state 318, a target state 320, a minimum-cost path 322, and a torsion angle sequence output 324.

FIG. 4 is a flowchart of an example protein folding method 400 including receiving a protein sequence 402, fixing cohesion quantum and ladder constants 404, assigning ladder indices to torsion and rotamer states 406, building a torsion-state graph 408, computing nonnegative edge costs 410, running a shortest-path search 412, extracting a torsion-angle pathway 414, reconstructing a three-dimensional structure 416, outputting a predicted structure and pathway 418, optionally computing ensemble averages 420, and ending the process 422.

FIG. 5 is a flowchart of an example RNA polymerase kinetics method 500 including receiving a nucleic acid sequence 502, assigning base ladder indices 504, computing position-dependent translocation rates 506, computing dwell times 508, computing a pause density profile 510, generating a pause histogram 512, registering a polymerase prefactor 514, outputting a kinetic profile and pause sites 516, and ending the process 518.

FIG. 6 is a block diagram of an example co-transcriptional simulation system 600 including a polymerase translocation module 602, an RNA folding module 604, a timing coupling interface 606, a transcription stepper 608, a nascent transcript buffer 610, a time manager 612, a secondary structure predictor 614, a protein folding module 616, a shared cohesion quantum module 618, a shared ladder index module 620, and an output module 622.

FIG. 7 is a block diagram of an example GPU-accelerated computing device 700 including a host processor 702, host memory 704, a GPU device 706, GPU memory 708, a host-device interconnect 710, a ladder assignment kernel 712, a graph build kernel 714, a shortest-path kernel 716, an ensemble averaging kernel 718, an RNA kinetics kernel 720, an output buffer 722, and a storage device 724.

FIG. 8 is a block diagram of an example FPGA-accelerated device 800 including a host interface 802, an input buffer 804, a ladder lookup table 806, a tunneling weight engine 808, a shortest-path solver pipeline 810, a Boltzmann averaging module 812, an RNA kinetics pipeline 814, an output buffer 816, a power manager 818, and a clock generator 820.

FIG. 9 is a block diagram of an example formal verification environment 900 including a theorem prover 902, a ladder energy formalization module 904, a tunneling formalization module 906, a pathway cost formalization module 908, an RNA kinetics formalization module 910, a constants formalization module 912, a proof artifacts repository 914, a build system 916, and a traceability and benchmark constants module 918.

FIG. 10 is a block diagram of an example benchmark workflow 1000 including a protein benchmark dataset 1002, an RNA kinetics benchmark dataset 1004, a predicted protein structure output 1006, a reference protein structure 1008, an RMSD evaluator 1010, a predicted pause histogram 1012, a reference pause histogram 1014, a histogram comparison metric 1016, a report generator 1018, and a performance metrics summary 1020.

FIG. 11 is a block diagram of an example drug ranking system 1100 including a target protein input 1102, a protein folding output 1104, a binding site identifier 1106, a docking module 1108, a binding site ladder assignment 1110, an energy difference calculator 1112, a compound ranking module 1114, a candidate selection module 1116, and an output report 1118.

FIG. 12 is a block diagram of an example cloud bio-folding service 1200 including a network API endpoint 1202, a request parser 1204, a job queue 1206, a compute cluster 1208, one or more GPU nodes 1210, one or more FPGA nodes 1212, a simulation engine instance 1214, results storage 1216, a response formatter 1218, and a client device 1220.

### DETAILED DESCRIPTION OF THE INVENTION

In general, the disclosed subject matter provides a biomolecular simulation architecture in which biomolecular states are represented using a fixed cohesion quantum and integer energy ladders instead of a conventional fitted force-field energy stack. In various embodiments, the architecture is configured to generate one or more of folding pathways, folded structures, translocation-rate profiles, dwell-time profiles, pause-density profiles, state-transition traces, ensemble statistics, compound rankings, and verification artifacts. In various embodiments, the disclosed architecture is implemented in software, firmware, hardware, or any combination thereof, including on one or more central processing units, graphics processing units, field-programmable gate arrays, application-specific accelerators, cloud computing nodes, edge devices, or distributed compute environments.

In interpretation, unless the context requires otherwise, the terms “comprising,” “including,” “having,” and similar open-ended terms are intended to be inclusive and not exclusive. In interpretation, reference to “one embodiment,” “an embodiment,” “some embodiments,” or similar phrasing indicates that a feature, structure, or operation described in connection with that embodiment can be included in at least one embodiment and is not necessarily required in all embodiments. In interpretation, singular terms can include plural referents and plural terms can include singular referents unless the context dictates otherwise. In interpretation, the recitation of alternatives is not intended to require mutual exclusivity unless expressly stated.

In terminology, the term “cohesion quantum” refers to a fixed energy quantum used as a step size for quantizing allowed biomolecular state energies. In terminology, the term “integer energy ladder” refers to a discrete energy set in which each allowed energy is represented as an integer multiple of the cohesion quantum. In terminology, the term “ladder index” or “ladder level index” refers to the integer value assigned to a biomolecular state for determining the state's ladder energy. In terminology, the term “state” refers to a machine-representable conformation, sub-conformation, sequence-dependent condition, or kinetic condition that is eligible for evaluation by the disclosed engine. In terminology, the term “pathway” refers to an ordered sequence of allowed states or allowed transitions between states. In terminology, the term “transition cost” refers to a nonnegative scalar associated with movement between states and suitable for accumulation along a path. In terminology, the term “tunneling weight” refers to a transition weight used to model a discrete transition tendency between ladder levels and, in some embodiments, is converted to an additive transition cost by a negative-log transformation.

In notation, a Recognition-Physics native cohesion quantum can be expressed as E_coh_RS=phi\hat{ }(−5), where phi is the golden ratio. In notation, phi can be expressed as phi=(1+sqrt(5))/2 and satisfies phi\hat{ }2=phi+1. In numerical embodiments, the same cohesion quantum can be represented in an eV-calibrated form as E_coh_eV having a fixed value, including about 0.090 eV in some embodiments. In ladder embodiments, an allowed ladder energy is expressed as E_n=n*E_coh_eV, where n is an integer and, in many embodiments, a nonnegative integer. In state embodiments, a state energy is expressed as E_state=n_l*E_coh_eV, where n_l is the ladder index assigned to the state.

In transition modeling, a tunneling weight can be expressed as W=exp(−(E_coh_eV)/(hbar*omega0)) for an allowed inter-level transition, and an additive cost can be expressed as C=−ln(W)=(E_coh_eV)/(hbar*omega0). In locked embodiments, a locking relation hbar*omega0=E_coh_eV is imposed so that the tunneling weight reduces to exp(−1) and the associated additive cost reduces to 1 for the locked step. In cost modeling, the disclosed engine can use state energy alone, differential energy, tunneling-derived cost alone, or a hybrid sum of those terms depending on the target use case, provided that the resulting edge costs supplied to a shortest-path solver are nonnegative in implementations that use solvers requiring nonnegative costs.

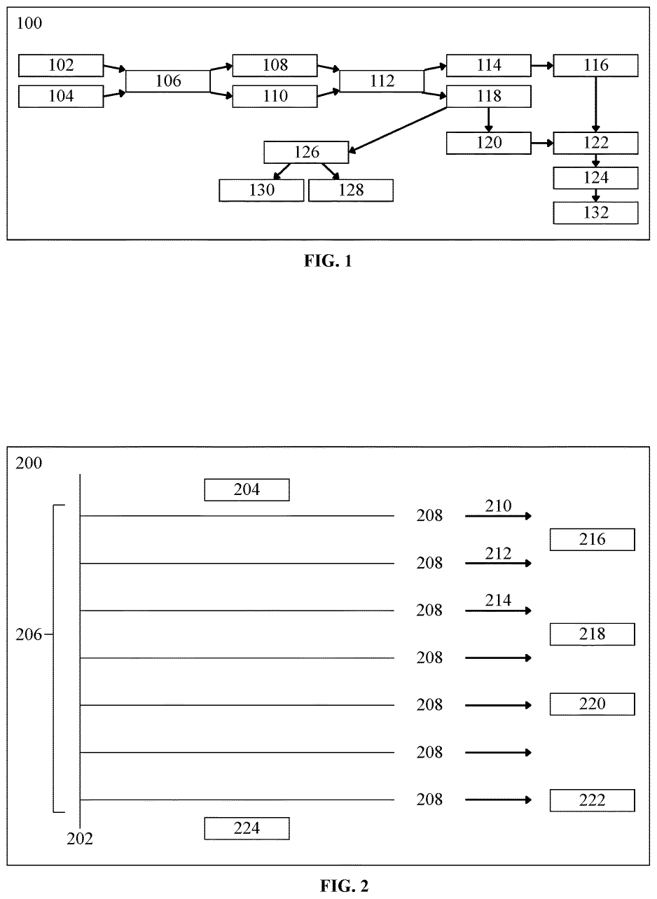

In system overview with reference to FIG. 1, a bio-folding system 100 includes an input interface 102 configured to receive sequence data 104. In some embodiments, the input interface 102 accepts amino-acid sequences, RNA sequences, DNA sequences, enzyme sequence descriptors, residue annotations, residue modifications, target identifiers, assay metadata, benchmark metadata, docking inputs, or combinations thereof. In some embodiments, the sequence data 104 is supplied as plain text, structured records, binary payloads, database records, application programming interface requests, or batch job inputs. In some embodiments, the input interface 102 validates character set, molecule type, sequence length, residue validity, nucleotide validity, and optional metadata completeness before allowing the sequence data 104 to advance to downstream modules.

In preprocessing with continued reference to FIG. 1, the bio-folding system 100 includes a preprocessing module 106 configured to normalize the sequence data 104 into internal machine representations. In some embodiments, the preprocessing module 106 strips unsupported symbols, resolves ambiguity codes, standardizes residue naming, standardizes nucleotide naming, tags modified positions, records chain boundaries, and generates one or more internal arrays, lookup structures, or graph seeds. In some embodiments, the preprocessing module 106 also determines whether the received input should be routed to protein-folding logic, RNA-kinetics logic, co-transcriptional logic, compound-ranking logic, or a combined workflow. In some embodiments, the preprocessing module 106 records normalization decisions in a trace log so that a later reviewer can distinguish a raw input from a normalized input used for actual computation.

In ladder assignment with continued reference to FIG. 1, the bio-folding system 100 includes a ladder index assignment module 108 configured to assign integer ladder indices to discrete biomolecular states. In some embodiments, the ladder index assignment module 108 receives discretized backbone-state candidates, rotamer-state candidates, nucleotide identities, base-step identities, sequence windows, local context labels, or enzyme-state labels and applies an objective mapping rule to produce one or more ladder indices. In some embodiments, the mapping rule is a fixed table stored in memory. In some embodiments, the mapping rule is a rule function defined by residue type, nucleotide type, region identifier, or molecule class. In some embodiments, the ladder index assignment module 108 emits a per-state ladder index record and a corresponding provenance record identifying which mapping rule caused the assignment.

In energy generation with continued reference to FIG. 1, the bio-folding system 100 includes an integer ladder energy module 110 configured to convert the ladder indices emitted by the ladder index assignment module 108 into state energies or edge-energy components. In some embodiments, the integer ladder energy module 110 stores E_coh_RS and E_coh_eV as fixed constants. In some embodiments, the integer ladder energy module 110 computes E_n values lazily when called by a downstream module. In some embodiments, the integer ladder energy module 110 precomputes a finite ladder-energy table for a supported range of n values and serves those values through direct lookup. In some embodiments, the integer ladder energy module 110 exposes both raw ladder energy and normalized ladder energy so that downstream modules can operate either in calibrated physical units or in dimensionless ladder units.

In graph generation with continued reference to FIG. 1, the bio-folding system 100 includes a torsion graph builder 112 configured to construct a graph representation of permitted folding states. In some embodiments, the torsion graph builder 112 constructs nodes for each residue position and each allowed discretized state at that position. In some embodiments, the torsion graph builder 112 stores each node as a tuple including at least a residue position, a state identifier, a ladder index, and one or more compatibility flags. In some embodiments, the torsion graph builder 112 constructs directed edges only between node pairs that satisfy adjacency rules, residue-order rules, steric rules, transition-span rules, or other admissibility rules. In some embodiments, the torsion graph builder 112 can operate on a fully materialized graph, a layered graph, or a streaming graph in which successor nodes are generated on demand.

In path solving with continued reference to FIG. 1, the bio-folding system 100 includes a shortest-path solver 114 configured to determine one or more minimum-cost paths through a graph built by the torsion graph builder 112. In some embodiments, the shortest-path solver 114 executes Dijkstra's algorithm when edge costs are guaranteed to be nonnegative. In some embodiments, the shortest-path solver 114 executes A* search when an admissible lower-bound heuristic is available. In some embodiments, the shortest-path solver 114 executes layered dynamic programming when the graph is acyclic or effectively acyclic due to position-layer ordering. In some embodiments, the shortest-path solver 114 emits not only a winning path but also one or more of a path-cost total, a predecessor table, a tie-resolution record, a queue trace, a frontier expansion record, and a ranked list of near-optimal alternatives.

In ensemble treatment with continued reference to FIG. 1, the bio-folding system 100 includes a Boltzmann averaging module 116 configured to compute ensemble-informed properties from state or pathway energies. In some embodiments, the Boltzmann averaging module 116 receives one or more candidate paths from the shortest-path solver 114 and applies Boltzmann weights using exp(−(E)/(k_B*T)) for a chosen temperature T. In some embodiments, the Boltzmann averaging module 116 receives state energies from the integer ladder energy module 110 and computes partition-function approximations. In some embodiments, the Boltzmann averaging module 116 produces one or more of occupancy values, expected observables, ranked state populations, temperature-response outputs, or pathway-weighted outputs. In some embodiments, the Boltzmann averaging module 116 is optional and can be bypassed when a user requests only a deterministic minimum-cost pathway.

In kinetics processing with continued reference to FIG. 1, the bio-folding system 100 includes an RNA kinetics module 118 configured to compute sequence-position-dependent kinetics using ladder indices derived from nucleic acid content. In some embodiments, the RNA kinetics module 118 computes k_RNA(i)=nu0*exp(−(n_i*E_coh_eV)/(k_B*T)), where nu0 is a registered polymerase prefactor and n_i is the ladder index assigned at position i. In some embodiments, the RNA kinetics module 118 computes dwell times tau(i)=1/k_RNA(i). In some embodiments, the RNA kinetics module 118 computes a normalized pause density sigma_RNA(i)=tau(i)/sum_j tau(j). In some embodiments, the RNA kinetics module 118 emits a full kinetic profile, a ranked pause list, a histogram, and a machine-readable trace indicating how each rate was computed from the underlying ladder assignments.

In co-transcriptional coupling with continued reference to FIG. 1, the bio-folding system 100 includes a co-transcriptional coupling module 120 configured to couple timing information from the RNA kinetics module 118 to a structure-formation model. In some embodiments, the co-transcriptional coupling module 120 exposes a current transcript length, a current dwell window, an accumulated elapsed time, and one or more pause indicators to a downstream folding engine. In some embodiments, the co-transcriptional coupling module 120 gates which states are eligible for evaluation based on the length of the nascent transcript available at a given simulation instant. In some embodiments, the co-transcriptional coupling module 120 therefore acts as a temporal constraint interface rather than merely a reporting interface.

In output generation with continued reference to FIG. 1, the bio-folding system 100 includes an output generator 122 configured to produce one or more outputs including predicted structures, state paths, torsion sequences, pause profiles, histograms, ranking reports, validation records, or verification artifacts. In some embodiments, the output generator 122 generates both human-readable and machine-actionable outputs. In some embodiments, the human-readable output includes formatted reports and summary tables, while the machine-actionable output includes state arrays, graph traces, coordinate files, serialized pathway objects, or structured application programming interface payloads. In some embodiments, the output generator 122 distinguishes inferred values from fixed constants and distinguishes final-state outputs from intermediate-state outputs.

In storage with continued reference to FIG. 1, the bio-folding system 100 includes storage 124 configured to store sequences, normalized inputs, state definitions, graph fragments, queue states, proof artifacts, benchmark metadata, output files, or combinations thereof. In some embodiments, the storage 124 includes volatile memory, nonvolatile memory, local file systems, object stores, database tables, or distributed storage services. In some embodiments, the storage 124 preserves traceability artifacts sufficient to reconstruct how a given output was generated from a given input under a given configuration.

In accelerator dispatch with continued reference to FIG. 1, the bio-folding system 100 includes an accelerator interface 126 configured to offload selected computations to a GPU accelerator 128, an FPGA accelerator 130, or both. In some embodiments, the accelerator interface 126 routes ladder assignment, graph construction, shortest-path evaluation, ensemble averaging, kinetics computation, or combinations thereof to an available accelerator based on configured policy. In some embodiments, the accelerator interface 126 distinguishes latency-sensitive tasks from throughput-sensitive tasks and assigns those tasks to different hardware back ends accordingly.

In external communications with continued reference to FIG. 1, the bio-folding system 100 includes a network interface 132 configured to communicate with remote clients, remote storage, cloud schedulers, benchmark repositories, or distributed execution nodes. In some embodiments, the network interface 132 receives jobs from a client, transmits results, fetches reference datasets, or synchronizes traceability records across systems. In some embodiments, the network interface 132 is omitted in stand-alone embodiments.

In ladder mechanics with reference to FIG. 2, an integer energy ladder diagram 200 conceptually illustrates how discrete state energies are represented. In some embodiments, an energy axis 202 represents increasing ladder energy. In some embodiments, an energy quantum step 204 corresponds to E_coh_eV and is the fixed spacing between adjacent ladder energy levels in the integer ladder representation. In some embodiments, a ladder level index 206 identifies a specific integer n value, and a ladder energy level 208 identifies the corresponding energy E_n. In some embodiments, one or more inter-level transition arrows 210 represent allowed transitions between adjacent or otherwise permitted levels. In some embodiments, the ladder representation is conceptual only. In other embodiments, the ladder representation is realized directly in data structures, lookup tables, transition matrices, or graph edge lists used during computation.

In state-energy assignment with reference to FIG. 2, a state energy annotation 224 indicates that the energy associated with a biomolecular state is determined from the assigned ladder index rather than from a fitted sum of many force-field terms. In some embodiments, the state energy is computed directly as E_state=n_l*E_coh_eV. In some embodiments, n_l is derived from a region identifier, rotamer identifier, nucleotide identity, enzyme state, or context-dependent label. In some embodiments, the disclosed engine allows multiple state classes to share the same cohesion-quantum ladder while using different ladder assignment rules. In some embodiments, this shared ladder architecture enables outputs from different modules to remain commensurate in the sense that they are all derived from the same fixed energy step.

In tunneling modeling with reference to FIG. 2, a tunneling weight annotation 212 indicates a multiplicative transition weight W associated with a state transition, and an additive transition cost annotation 214 indicates a corresponding additive cost C. In some embodiments, W and C are both stored. In some embodiments, only C is stored after a one-time transformation. In some embodiments, a locking relation annotation 216 indicates that hbar*omega0 is set equal to E_coh_eV so that W becomes exp(−1) and C becomes 1. In some embodiments, the locked representation simplifies edge generation and reduces per-edge arithmetic overhead. In some embodiments, the unlocked representation is retained when a user or implementation requires a nonconstant transition cost tied to a characteristic frequency term.

In thermal weighting with reference to FIG. 2, a Boltzmann factor annotation 218 and a temperature parameter annotation 220 illustrate that ladder energies can be converted to thermal weights for ensemble or occupancy calculations. In some embodiments, a temperature parameter is user supplied. In some embodiments, the temperature parameter is read from assay metadata or benchmark metadata. In some embodiments, multiple temperatures are evaluated in a batch mode for sensitivity analysis. In some embodiments, the disclosed engine stores the temperature actually used for a run as part of the provenance record.

In ladder admissibility with reference to FIG. 2, an integer constraint annotation 222 indicates that the ladder level index is constrained to integer values and, in many embodiments, to nonnegative integer values. In some embodiments, zero corresponds to a ground or minimum ladder state used as a native, near-native, or baseline reference. In some embodiments, higher indices correspond to progressively higher ladder costs and therefore lower preference under a minimum-cost or Boltzmann-weighted treatment. In some embodiments, a maximum supported ladder index is configured per model family or per implementation to bound memory usage and graph size.

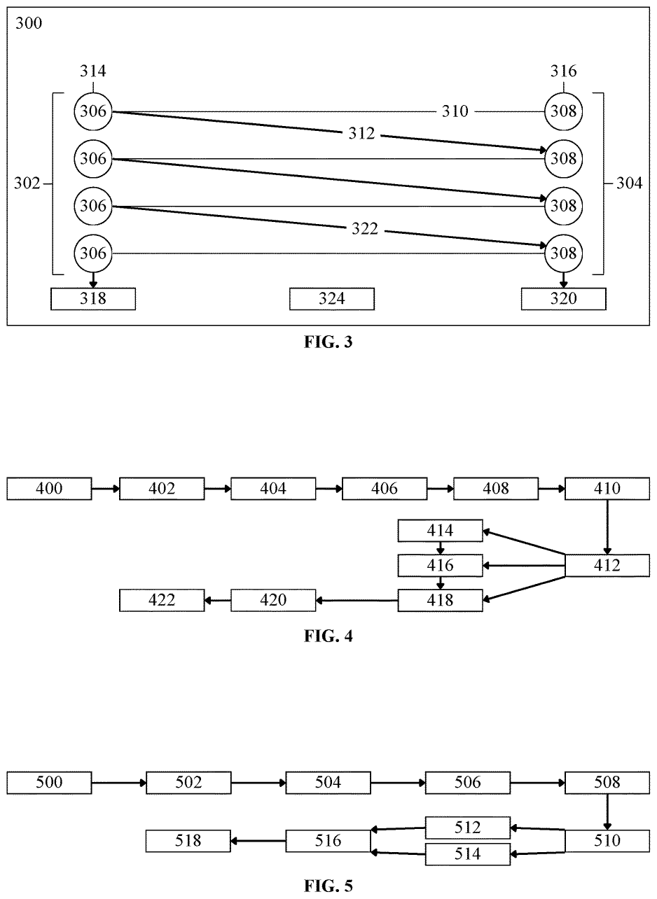

In protein-state graph construction with reference to FIG. 3, a torsion-state graph 300 represents permitted folding states as graph nodes and permitted transitions as graph edges. In some embodiments, residue i 302 and residue i+1 304 identify adjacent residues in sequence order. In some embodiments, a torsion state node for residue i 306 represents one allowed state for residue i, and a torsion state node for residue i+1 308 represents one allowed state for residue i+1. In some embodiments, an adjacency edge 310 is created only when the two corresponding states satisfy local transition admissibility rules. In some embodiments, admissibility rules include sequence adjacency, allowed torsion-span change, allowed region compatibility, steric exclusion tests, residue-type constraints, or combinations thereof.

In edge-cost assignment with reference to FIG. 3, an edge cost label 312 indicates the transition cost associated with an adjacency edge 310. In some embodiments, the edge cost label 312 equals the destination-state ladder energy. In some embodiments, the edge cost label 312 equals a difference term derived from source and destination ladder indices. In some embodiments, the edge cost label 312 includes an additive tunneling-derived cost. In some embodiments, the edge cost label 312 includes both a structural-state component and a transition component, provided that the total remains nonnegative in implementations that require nonnegative edge costs. In some embodiments, the edge cost label 312 is computed during graph construction. In some embodiments, the edge cost label 312 is computed lazily during solver expansion.

In backbone discretization with reference to FIG. 3, a Ramachandran region discretization 314 indicates that continuous backbone dihedral space can be discretized into a finite number of permitted regions. In some embodiments, six regions are used. In one illustrative embodiment, the six regions are labeled R0, R1, R2, R3, R4, and R5 and are mapped respectively to ladder indices n=0, 1, 2, 3, 4, and 5. In some embodiments, a candidate backbone state for residue i is encoded as a tuple (i, region identifier, n), and the state energy is computed as E_state=n*E_coh_eV. In some embodiments, the region boundaries and the region-to-index mapping are stored in the rule table. In some embodiments, more or fewer regions are used. In some embodiments, the region boundaries are fixed beforehand and stored in a rule table. In some embodiments, each region is associated with a ladder index through a ladder index assignment rule 316. In some embodiments, the same residue type can map to different permitted region subsets depending on residue-specific constraints, although all such variants still produce integer ladder indices.

In path definition with reference to FIG. 3, a source state 318 identifies a starting condition and a target state 320 identifies a terminating condition. In some embodiments, the source state 318 corresponds to a high-index unfolded or less ordered state. In some embodiments, the target state 320 corresponds to a low-index native or near-native state. In some embodiments, the shortest-path solver determines a minimum-cost path 322 connecting the source state 318 and the target state 320. In some embodiments, a torsion angle sequence output 324 is then derived from the ordered node list on the minimum-cost path 322. In some embodiments, the torsion angle sequence output 324 is the immediate precursor to three-dimensional coordinate reconstruction.

In state encoding for protein embodiments, each node can be encoded as a tuple containing at least a residue index, a backbone-region identifier, an optional side-chain rotamer identifier, a ladder index, and a state identifier. In some embodiments, optional fields include a predecessor pointer, a local energy cache, an admissibility mask, a residue-class tag, or a structure-fragment identifier. In some embodiments, the torsion graph builder 112 stores node tuples in contiguous memory for accelerator compatibility. In some embodiments, the torsion graph builder 112 stores edge lists in compressed sparse row format, layered adjacency tables, or streamed successor generators.

In side-chain treatment for protein embodiments, side-chain states can be integrated directly into the node definition or treated as a subordinate local-state calculation. In some embodiments, a combined node state includes both a backbone-region assignment and a side-chain rotamer assignment. In some embodiments, side-chain assignments are computed after a backbone path is selected. In some embodiments, side-chain assignments are solved by a second-stage graph search, by local optimization, or by rule-based selection. In some embodiments, side-chain states receive ladder indices derived from chi-angle bins, rotamer-library classes, or residue-specific rotamer categories.

In structure reconstruction for protein embodiments, the disclosed engine converts an extracted pathway into coordinates using internal-coordinate reconstruction. In some embodiments, bond lengths and bond angles are fixed to canonical values for the relevant residue types. In some embodiments, torsion angles are drawn directly from the selected discrete state definitions. In some embodiments, chirality rules, peptide-bond planarity rules, closure checks, clash checks, or combinations thereof are applied during reconstruction. In some embodiments, reconstruction failures cause the engine to fall back to the next-ranked path, to a local repair step, or to a flagged output indicating that the pathway was valid in graph terms but failed coordinate realization constraints.

In method flow with reference to FIG. 4, a protein folding method 400 begins at start 400. In some embodiments, receiving a protein sequence 402 includes receiving a primary sequence and, optionally, one or more of chain metadata, modified residue flags, or target constraints. In some embodiments, fixing cohesion quantum and ladder constants 404 includes loading E_coh_RS, loading or deriving E_coh_eV, selecting a temperature if ensemble calculations are requested, and selecting locked or unlocked tunneling-cost treatment. In some embodiments, assigning ladder indices to torsion and rotamer states 406 includes generating the candidate discrete state library for each residue and assigning each candidate state a ladder index according to the mapping rules in force for the current run.

In graph preparation with continued reference to FIG. 4, building a torsion-state graph 408 includes creating node sets per residue position, determining permitted transitions between adjacent layers, and associating each permitted transition with one or more cost components. In some embodiments, computing nonnegative edge costs 410 includes calculating destination-state costs, differential costs, tunneling-step costs, or hybrids thereof and verifying that the resulting edge values satisfy solver requirements. In one illustrative embodiment, a destination-state cost for a transition into a candidate state is n_dest*E_coh_eV, a differential cost is max(0, n_dest−n_src)*E_coh_eV, and a locked tunneling-step cost adds 1 for each permitted adjacent ladder step. In some embodiments, a hybrid edge cost is formed as the sum of the destination-state cost and the tunneling-step cost, and any candidate transition producing a negative value or violating a permitted-transition rule is rejected before solver execution. In some embodiments, a pre-solver validation pass confirms that each node layer is populated, that at least one source-to-target path exists, and that no unsupported negative edge values are present.

In solver execution with continued reference to FIG. 4, running a shortest-path search 412 includes initializing a source frontier, expanding eligible nodes, updating tentative costs, tracking predecessor relationships, and terminating when a target-state condition is satisfied or when no valid path exists. In some embodiments, extracting a torsion-angle pathway 414 includes reconstructing the winning node chain from predecessor records and converting the node chain into a sequence of explicit torsion-state definitions. In some embodiments, reconstructing a three-dimensional structure 416 includes generating Cartesian coordinates from that torsion-state sequence. In some embodiments, outputting a predicted structure and pathway 418 includes storing both a final structure and the path record. In some embodiments, optionally computing ensemble averages 420 includes evaluating a selected number of alternative low-cost paths under Boltzmann weighting. The process then reaches end 422.

In solver failure handling for the protein folding method 400, if the graph contains no valid path from the configured source to the configured target, the system can emit an explicit no-path condition rather than silently returning a partial result. In some embodiments, the system then retries using a relaxed compatibility policy, an expanded state library, a different source-state definition, or a different target-state definition. In some embodiments, all such fallback decisions are logged separately from the final selected computational path so that a reviewer can distinguish the primary run configuration from a fallback configuration.

In tie handling for the protein folding method 400, multiple equal-cost paths can arise. In some embodiments, the shortest-path solver 114 resolves equal-cost candidates by a deterministic secondary rule, such as lower cumulative high-index count, lower lexicographic state identifier, lower residue-wise variance, or earlier discovery order under a fixed queue policy. In some embodiments, all tied candidates are preserved and forwarded to the Boltzmann averaging module 116. In some embodiments, preserving equal-cost alternatives strengthens downstream ensemble treatment and reduces arbitrary path collapse.

In data outputs for the protein folding method 400, the engine can emit a coordinate file, a pathway record, a node trace, an edge trace, a per-residue state sequence, a ladder-index sequence, a path-cost decomposition, a reconstruction-status record, and a provenance record. In some embodiments, the provenance record identifies the exact mapping tables, constants, temperature, solver type, and fallback policies used for the run. In some embodiments, this allows later auditor review without reinterpreting the raw code base.

In RNA kinetics operation with reference to FIG. 5, an RNA polymerase kinetics method 500 begins at start 500. In some embodiments, receiving a nucleic acid sequence 502 includes receiving a DNA template sequence, an RNA sequence, a promoter-associated region, a transcript segment, a genome segment, or a benchmark sequence set. In some embodiments, the received sequence is tagged with polymerase identity, temperature, assay source, strand direction, reference coordinate metadata, or combinations thereof. In some embodiments, the system validates that the sequence contains supported base symbols and converts any supported ambiguity code into a deterministic working representation before downstream kinetic computation begins.

In ladder assignment for RNA kinetics with continued reference to FIG. 5, assigning base ladder indices 504 includes mapping each nucleotide position to a ladder index according to an objective base-identity rule. In some embodiments, adenine maps to 0, thymine or uracil maps to 1, guanine maps to 2, and cytosine maps to 3. In some embodiments, the mapping is stored as a fixed lookup table. In some embodiments, the mapping is adjusted only by selecting among a finite predefined rule set rather than by fitting a continuous parameter family. In some embodiments, the resulting ladder-index array is stored as a position-indexed vector n_i for subsequent rate calculation.

In rate generation for RNA kinetics with continued reference to FIG. 5, computing position-dependent translocation rates 506 includes applying a ladder-index-based exponential rate law. In some embodiments, the rate at position i is computed as k_RNA(i)=nu0*exp(−(n_i*E_coh_eV)/(k_B*T)). In some embodiments, nu0 is a polymerase-specific prefactor stored in a registry associated with polymerase species or polymerase class. In some embodiments, the rate law is applied independently at each sequence position. In some embodiments, neighboring positions, local windows, pause-memory flags, or context states can be incorporated as long as the output remains traceable to the fixed cohesion quantum and integer-ladder architecture.

In dwell-time calculation for RNA kinetics with continued reference to FIG. 5, computing dwell times 508 includes converting each position-dependent rate to a dwell interval, such as tau(i)=1/k_RNA(i). In some embodiments, a dwell-time array is stored in sequence order for later normalization and reporting. In some embodiments, if a rate underflows numerical precision or falls below a configured floor, the system clips the value to a minimum supported positive rate and records the clipping event in a trace log. In some embodiments, if a rate overflows due to an unsupported configuration, the system either rejects the run or applies a bounded fallback transformation rather than silently producing invalid pause outputs.

In pause normalization for RNA kinetics with continued reference to FIG. 5, computing a pause density profile 510 includes normalizing dwell times across the evaluated sequence window. In some embodiments, sigma_RNA(i)=tau(i)/sum_j tau(j). In some embodiments, the disclosed engine uses only positions within a selected window when calculating the denominator. In some embodiments, the engine uses the full evaluated sequence. In some embodiments, masked positions are excluded from both numerator and denominator. In some embodiments, the resulting normalized values sum to 1 over the included position set and can be rendered as a position-resolved pause density profile.

In histogram generation with continued reference to FIG. 5, generating a pause histogram 512 includes converting the pause density profile to a histogram representation, peak list, ranked pause-site list, smoothed curve, or thresholded event map. In some embodiments, the engine preserves the raw unsmoothed values and stores any smoothed or thresholded representation separately so that observation is distinguishable from post-processing. In some embodiments, registering a polymerase prefactor 514 includes storing or retrieving a previously defined nu0 value for the active polymerase. In some embodiments, outputting a kinetic profile and pause sites 516 includes returning rates, dwell times, normalized pause values, rank-ordered peaks, and coordinate labels before reaching end 518.

In RNA kinetics fallback behavior, the disclosed engine can support multiple policy layers while preserving the disclosed ladder logic. In some embodiments, if a nucleotide symbol is unsupported, the engine rejects the sequence. In some embodiments, if a nucleotide symbol is ambiguous but supported by policy, the engine resolves it according to a deterministic priority rule, such as selecting the lowest-index compatible base, the highest-index compatible base, or a configured canonical substitute. In some embodiments, all such substitutions are separately logged so that a later user can distinguish native sequence content from policy-resolved content.

In polymerase selection for RNA kinetics, the disclosed engine can operate with multiple polymerase embodiments without abandoning the common architecture. In some embodiments, an Escherichia coli RNA polymerase embodiment uses a stored nu0 value associated with E. coli RNAP. In some embodiments, a T7 RNA polymerase embodiment uses a stored nu0 value associated with T7 RNAP. In some embodiments, a yeast polymerase II embodiment uses a stored nu0 value associated with Pol II. In some embodiments, the engine does not refit nu0 per sequence once a selected polymerase registry entry is chosen for a run, thereby maintaining sequence-level determinism for a given polymerase configuration.

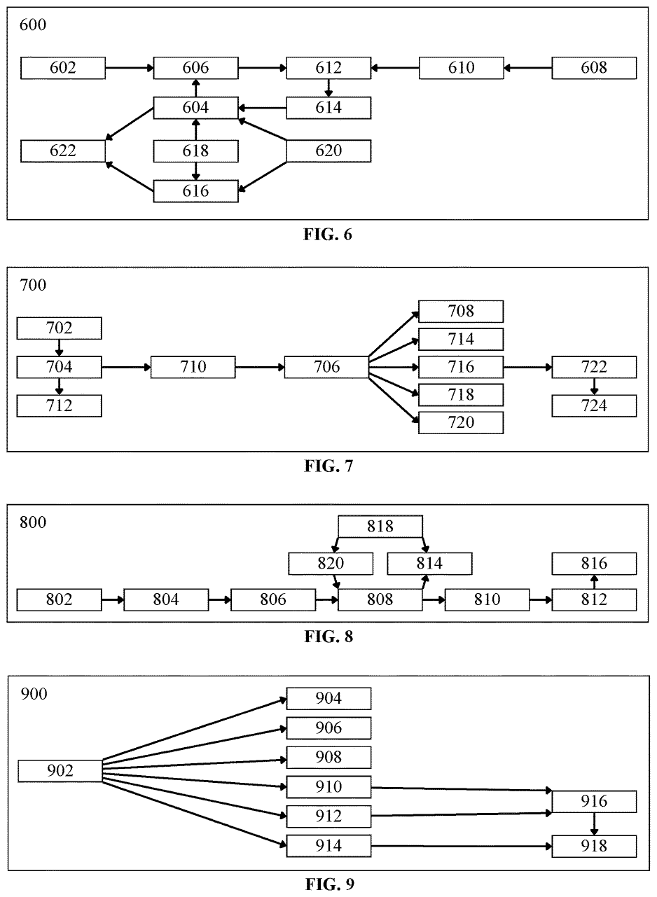

In co-transcriptional simulation with reference to FIG. 6, a co-transcriptional simulation system 600 includes a polymerase translocation module 602 and an RNA folding module 604 connected through a timing coupling interface 606. In some embodiments, the polymerase translocation module 602 computes sequence-position-dependent timing using the ladder-based kinetics described above. In some embodiments, the RNA folding module 604 evaluates structure accessibility for the current nascent transcript. In some embodiments, the timing coupling interface 606 converts polymerase dwell behavior into a structure-formation time window that constrains which states are considered reachable before the next transcription increment.

In transcription stepping with reference to FIG. 6, a transcription stepper 608 advances the simulated polymerase position along the sequence. In some embodiments, the transcription stepper 608 increments by one nucleotide at a time. In some embodiments, the transcription stepper 608 can use batched increments for accelerated simulation while still preserving a cumulative dwell-time record. In some embodiments, each step causes the nascent transcript buffer 610 to expose a longer transcript prefix to the RNA folding module 604. In some embodiments, the timing coupling interface 606 updates a remaining dwell interval or total elapsed interval after each step.

In nascent-state management with reference to FIG. 6, the nascent transcript buffer 610 stores the currently available transcript portion and, in some embodiments, one or more intermediate buffer states. In some embodiments, the buffer includes a sequence prefix, an index of exposed positions, and one or more metadata flags indicating pause status or boundary conditions. In some embodiments, the time manager 612 maintains absolute simulated time, relative dwell time, cumulative pause time, and event timestamps. In some embodiments, the time manager 612 is also used to synchronize folding outputs with transcription positions in a time-resolved report.

In secondary-structure treatment with reference to FIG. 6, a secondary structure predictor 614 evaluates possible RNA structure states available to the current nascent transcript. In some embodiments, the secondary structure predictor 614 uses an integer-ladder representation of base-pair formation states. In some embodiments, the secondary structure predictor 614 uses a dynamic-programming architecture adapted to ladder states rather than to fitted thermodynamic tables. In some embodiments, the secondary structure predictor 614 produces one or more candidate secondary structures, structural transitions, pairing states, or pathway states for the currently exposed transcript length.

In shared architecture with reference to FIG. 6, a protein folding module 616 can coexist with the RNA folding module 604 within the same broader simulation framework. In some embodiments, a shared cohesion quantum module 618 provides a common fixed energy step to both protein and RNA processing paths. In some embodiments, a shared ladder index module 620 provides a common assignment framework or a common assignment service with molecule-type-specific rule tables. In some embodiments, an output module 622 combines timing outputs and structure outputs into a unified report, thereby allowing a user to see not only which structure is predicted but also when that structure becomes accessible during transcription.

In temporal coupling logic for co-transcriptional embodiments, the disclosed engine distinguishes observation from control. In some embodiments, pause timing is merely observed and reported. In some embodiments, pause timing actively controls state eligibility by limiting or enabling transitions in the RNA folding module 604. In some embodiments, a longer dwell window at a position permits evaluation of additional state transitions or additional folding rounds before the transcription stepper 608 advances. In some embodiments, this creates a direct technical coupling between kinetic output and folding-state accessibility, rather than a mere side-by-side display of unrelated analyses.

In GPU implementation with reference to FIG. 7, a GPU-accelerated computing device 700 includes a host processor 702 and host memory 704 coupled to a GPU device 706 and GPU memory 708 through a host-device interconnect 710. In some embodiments, the host processor 702 receives inputs, prepares normalized work units, and dispatches one or more kernels to the GPU device 706. In some embodiments, the GPU memory 708 stores sequence arrays, node arrays, edge arrays, cost arrays, output buffers, or combinations thereof. In some embodiments, the host-device interconnect 710 supports direct memory transfer, pinned-memory transfer, or staged transfer according to implementation choice.

In ladder-parallel processing with reference to FIG. 7, a ladder assignment kernel 712 computes ladder indices for residues, nucleotides, or candidate states in parallel. In some embodiments, the ladder assignment kernel 712 reads sequence symbols from a compact input array and emits integer ladder values to a device-resident output array. In some embodiments, the ladder assignment kernel 712 also emits rule identifiers or provenance flags. In some embodiments, the ladder assignment kernel 712 is fused with a downstream kernel to reduce global-memory writes.

In graph construction on GPU with reference to FIG. 7, a graph build kernel 714 generates graph nodes, edge lists, or both. In some embodiments, the graph build kernel 714 uses a residue-layer model in which threads construct local adjacency for specific residue transitions. In some embodiments, the graph build kernel 714 computes edge costs while creating the adjacency relation. In some embodiments, the graph build kernel 714 stores the resulting graph in compressed sparse row format, edge-tuple format, or tiled edge-block format. In some embodiments, invalid transitions are filtered before emission to conserve memory and solver bandwidth.

In GPU path solving with reference to FIG. 7, a shortest-path kernel 716 evaluates path costs on the GPU device 706. In some embodiments, the shortest-path kernel 716 implements a frontier-based relaxation method adapted to the graph shape produced by the graph build kernel 714. In some embodiments, the shortest-path kernel 716 is optimized for layered sparse graphs and avoids generic all-pairs operations. In some embodiments, the shortest-path kernel 716 returns predecessor indices, final costs, and target-state identifiers. In some embodiments, a host-side pass reconstructs the winning path from the predecessor data. In some embodiments, device-side reconstruction is also supported.

In GPU ensemble and kinetics processing with reference to FIG. 7, an ensemble averaging kernel 718 computes thermal weights, partial sums, or normalized observables in parallel. In some embodiments, an RNA kinetics kernel 720 computes position-specific rates, dwell times, pause densities, or histogram bins across many sequence positions or across many sequences in a batch. In some embodiments, an output buffer 722 stores intermediate or final results pending return to the host processor 702. In some embodiments, a storage device 724 stores persisted outputs, benchmark traces, or reproducibility artifacts.

In GPU precision policy, mixed precision can be used where appropriate. In some embodiments, integer ladder indices are stored in integer arrays, ladder energies are stored in FP32 arrays, and selected path accumulators or exponential computations are performed in FP32 or FP64 depending on the numeric-stability policy for the run. In some embodiments, if an implementation uses FP16 in intermediate steps, the system preserves a higher-precision accumulation path for cost totals or exponentials to reduce unacceptable numeric distortion. In some embodiments, all precision choices are recorded as run metadata because numeric precision affects reproducibility boundaries.

In FPGA implementation with reference to FIG. 8, an FPGA-accelerated device 800 includes a host interface 802, an input buffer 804, a ladder lookup table 806, a tunneling weight engine 808, a shortest-path solver pipeline 810, a Boltzmann averaging module 812, an RNA kinetics pipeline 814, an output buffer 816, a power manager 818, and a clock generator 820. In some embodiments, the FPGA-accelerated device 800 is used when deterministic streaming execution, lower power consumption, or a fixed-function deployment profile is preferred.

In ladder lookup on FPGA with reference to FIG. 8, the ladder lookup table 806 stores ladder energies for a supported range of integer ladder values. In some embodiments, the ladder lookup table 806 stores energies directly in calibrated units. In some embodiments, the ladder lookup table 806 stores dimensionless step counts and a downstream stage applies the fixed cohesion-quantum scale. In some embodiments, the ladder lookup table 806 also stores tunneling-cost constants for locked embodiments. In some embodiments, table lookup reduces repeated arithmetic and improves deterministic latency.

In tunneling and path pipelines on FPGA with reference to FIG. 8, the tunneling weight engine 808 computes transition weights or additive costs for permitted transitions. In some embodiments, the tunneling weight engine 808 is bypassed in locked embodiments where the cost per adjacent step is constant and can be supplied directly. In some embodiments, the shortest-path solver pipeline 810 processes layered graph transitions as a streaming relaxation pipeline. In some embodiments, the shortest-path solver pipeline 810 supports one or more residue layers per cycle group. In some embodiments, the pipeline retains predecessor traces in on-chip memory or staged off-chip memory for later path reconstruction.

In thermal and kinetic pipelines on FPGA with reference to FIG. 8, the Boltzmann averaging module 812 computes weighted sums or normalization factors for ensemble outputs, while the RNA kinetics pipeline 814 computes rates, dwell values, or normalized pause values. In some embodiments, the output buffer 816 stores results before host transfer. In some embodiments, the power manager 818 enforces operating constraints and the clock generator 820 supplies synchronized timing for the pipeline stages. In some embodiments, the FPGA implementation is particularly advantageous for continuous batch processing of many sequences under a fixed ruleset.

In formal verification with reference to FIG. 9, a formal verification environment 900 includes a theorem prover 902 and one or more specialized formalization modules. In some embodiments, a ladder energy formalization module 904 proves nonnegativity of ladder energies, monotonicity with increasing ladder index, and uniform spacing between adjacent ladder levels. In some embodiments, a tunneling formalization module 906 proves positivity and boundedness properties for tunneling weights. In some embodiments, a pathway cost formalization module 908 proves that accumulated path costs remain nonnegative under the selected cost law. In some embodiments, an RNA kinetics formalization module 910 proves positivity of computed rates and consistency of kinetic definitions with shared ladder constants.

In formal constants and artifacts with reference to FIG. 9, a constants formalization module 912 proves or checks properties associated with the fixed cohesion quantum and related identities, a proof artifacts repository 914 stores proof outputs, a build system 916 compiles and validates proof assets, and a traceability and benchmark constants module 918 stores benchmark constants or target values used to connect implementation statements with specific tested configurations. In some embodiments, the formal verification environment 900 is integrated into a continuous-integration pipeline. In some embodiments, it is executed only during validation or release. In some embodiments, proof success is stored separately from runtime success so that proof of a general property is distinguishable from the outcome of a specific simulation run.

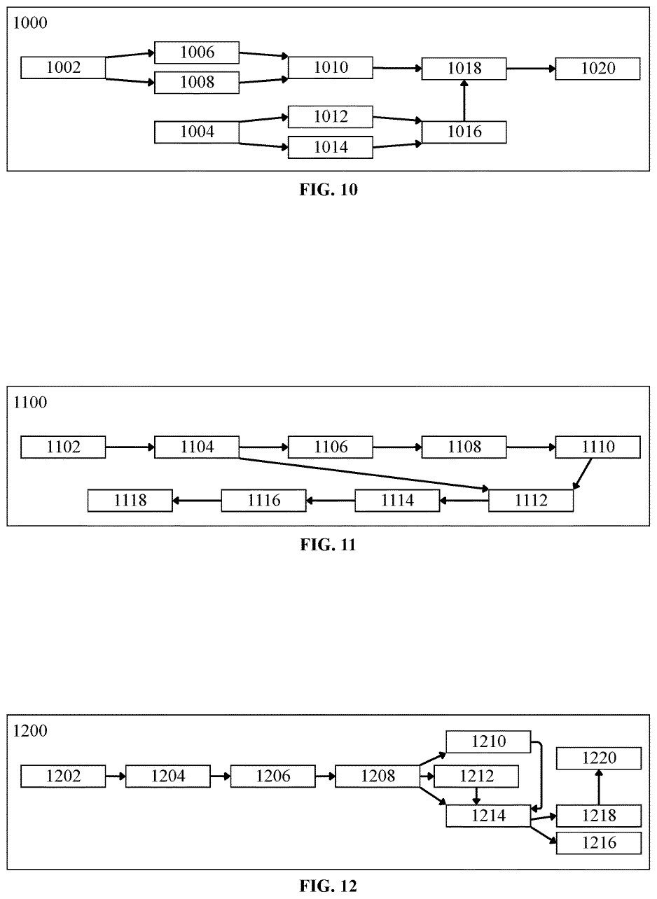

In benchmark evaluation with reference to FIG. 10, a benchmark workflow 1000 includes a protein benchmark dataset 1002 and an RNA kinetics benchmark dataset 1004. In some embodiments, a predicted protein structure output 1006 is compared with a reference protein structure 1008 by an RMSD evaluator 1010. In some embodiments, a predicted pause histogram 1012 is compared with a reference pause histogram 1014 by a histogram comparison metric 1016. In some embodiments, a report generator 1018 produces human-readable and machine-readable evaluation outputs, and a performance metrics summary 1020 records runtime, throughput, memory, accuracy, or energy-use measures.

In benchmark policy, the disclosed engine can distinguish benchmark outputs from guaranteed outputs. In some embodiments, an RMSD value, pause-agreement value, throughput value, or power value is reported only as a result observed under a stated configuration. In one illustrative benchmark policy, a protein benchmark case is recorded as satisfying a stated structural target when RMSD is less than or equal to 2.0 angstroms, and an RNA kinetics benchmark case is recorded as satisfying a stated histogram-agreement target when histogram error under the selected comparison metric is within plus or minus 5 percent. In some embodiments, such thresholds are stored as benchmark-policy metadata rather than as tunable simulation parameters. In some embodiments, benchmark and validation values described herein are exemplary observed results under stated datasets, hardware configurations, and rulesets, and are not required features of every embodiment unless expressly recited in a particular claim. In some embodiments, the engine stores the hardware identity, dataset identity, solver identity, and constants identity associated with that reported result. In some embodiments, such records improve reproducibility and reduce ambiguity about whether a reported benchmark is theoretical, estimated, or actually measured.

In drug-ranking embodiments with reference to FIG. 11, a drug ranking system 1100 includes a target protein input 1102, a protein folding output 1104, a binding site identifier 1106, a docking module 1108, a binding site ladder assignment 1110, an energy difference calculator 1112, a compound ranking module 1114, a candidate selection module 1116, and an output report 1118. In some embodiments, the target protein input 1102 is a raw sequence and the protein folding output 1104 is generated by the same disclosed folding architecture. In some embodiments, the target protein input 1102 includes an already known or externally supplied structure.

In binding-site ladder treatment with reference to FIG. 11, the binding site identifier 1106 selects a region of interest, and the docking module 1108 generates one or more candidate poses for candidate compounds. In some embodiments, the binding site ladder assignment 1110 assigns ladder indices to binding-site residues in the unbound and bound conditions. In some embodiments, the energy difference calculator 1112 computes DeltaE=(sum_i n_i_bound−sum_i n_i_unbound)*E_coh_eV. In some embodiments, the compound ranking module 1114 sorts candidate compounds by DeltaE or by a related ladder-derived score. In some embodiments, the candidate selection module 1116 selects a top subset for downstream experimental validation. In some embodiments, the output report 1118 preserves the difference between the machine-actionable ranking and any human-readable explanation.

In cloud deployment with reference to FIG. 12, a cloud bio-folding service 1200 includes a network API endpoint 1202, a request parser 1204, a job queue 1206, a compute cluster 1208, one or more GPU nodes 1210, one or more FPGA nodes 1212, a simulation engine instance 1214, results storage 1216, a response formatter 1218, and a client device 1220. In some embodiments, the network API endpoint 1202 receives a request specifying a molecule type, sequence content, desired output mode, or benchmark mode. In some embodiments, the request parser 1204 validates the request and rejects unsupported request types before a job is queued.

In cloud scheduling with reference to FIG. 12, the job queue 1206 can schedule jobs according to latency policy, throughput policy, benchmark policy, or hardware-availability policy. In some embodiments, the compute cluster 1208 includes heterogeneous compute resources, including the GPU nodes 1210 and the FPGA nodes 1212. In some embodiments, the scheduler assigns a given job to a simulation engine instance 1214 configured for the requested molecule type and output mode. In some embodiments, results storage 1216 stores intermediate and final outputs, and the response formatter 1218 returns a structured result to the client device 1220. In some embodiments, the cloud embodiment supports synchronous responses for smaller jobs and asynchronous retrieval for larger jobs.

In molecule-specific alternative embodiments, membrane proteins can be modeled by assigning ladder indices to membrane-insertion states, lipid-facing states, or transmembrane region states while remaining within the same fixed-cohesion-quantum architecture. In some embodiments, protein-protein docking is modeled by building a combined interface-state graph in which interface residues or interface patches receive ladder indices and low-cost interface arrangements are searched over a discrete graph. In some embodiments, RNA secondary structure prediction is implemented with ladder-indexed pairing states and dynamic programming over those states. In some embodiments, enzyme design is implemented by evaluating how candidate mutations alter local ladder assignments and thereby alter predicted transition or kinetic outputs.

In nanopore embodiments, a nucleotide in a sensing region can be assigned a ladder state and mapped to a predicted ionic-current category or current value using a ladder-index-informed signal model. In some embodiments, the disclosed engine therefore outputs both biomolecular structural information and sensor-prediction information. In some embodiments, a quantum-classical hybrid deployment partitions work such that tunneling-dominated transitions are delegated to a specialized hardware path while graph management or Boltzmann weighting remains on classical hardware. In some embodiments, such partitioning is controlled by a deterministic scheduler that records which portions of a result were produced by which compute path.

In implementation boundaries and claim support, the disclosed engine distinguishes optional features from required features. In some embodiments, the fixed cohesion quantum and integer energy ladder are core features. In some embodiments, shortest-path solving is core for graph-path embodiments. In some embodiments, a first claim set is directed to a protein-folding pathway engine in which the generated output comprises a folded structure and an ordered state pathway, and a second claim set is directed to a nucleic-acid kinetics engine in which the generated output comprises position-dependent rates, dwell times, and a normalized pause-density profile. In some embodiments, formal verification, cloud deployment, accelerator execution, docking integration, ensemble averaging, and co-transcriptional timing are optional features. In some embodiments, the disclosed specification provides support for independent claims drawn to the core ladder architecture and dependent claims drawn to one or more optional refinements or deployment models.

In data-governance and reproducibility embodiments, the engine can store version identifiers for mapping rules, graph policies, solver logic, and constants packages. In some embodiments, a result is tagged with a run identifier, a ruleset identifier, a hardware identifier, and a benchmark identifier. In some embodiments, this permits exact replay. In some embodiments, exact replay is defined as re-execution under the same input, same constants, same mapping tables, same solver policy, and same precision policy. In some embodiments, replayability is an important technical effect because it distinguishes the disclosed deterministic ladder architecture from opaque or weakly reproducible parameter-tuned workflows.

In definitional discipline, the disclosed engine distinguishes storage from validation, retrieval from acceptance, detection from action, and observation from control. In some embodiments, storing a sequence or constant does not imply that the value has been validated. In some embodiments, detecting a possible state or pause site does not itself trigger ranking, output, or control unless a downstream decision rule is applied. In some embodiments, retrieving a historical benchmark does not imply that the benchmark is used as a current state. In some embodiments, these distinctions reduce ambiguity in claim interpretation and implementation review.

In scope, the particular values, mappings, discretizations, graph structures, solver choices, hardware architectures, proof environments, and output formats described herein are illustrative and non-limiting unless expressly recited otherwise. In some embodiments, more or fewer state classes can be used, more or fewer graph layers can be used, and more or fewer output modes can be generated, provided that the implementation retains the disclosed architecture of a fixed cohesion quantum, integer-ladder state representation, and explicit state-transition computation. In some embodiments, the disclosed subject matter is not limited to any specific programming language, database type, file format, instruction set, operating system, or cloud provider.

In claim interpretation, no claimed element is intended to invoke a special means-plus-function construction unless expressly recited using language traditionally associated with that doctrine and unless no sufficient structure is otherwise recited. In claim interpretation, structural references to modules, engines, pipelines, tables, interfaces, buffers, nodes, graphs, kernels, or repositories denote concrete machine-implemented or machine-implementable structures and workflows as described herein. In claim interpretation, where a method step is described in a sequence, that sequence is illustrative unless the claim language later requires a specific order or unless a particular order is logically necessary.

In concluding description, the disclosed architecture provides a concrete technical mechanism for representing biomolecular behavior through fixed-step integer ladder energies, objective transition rules, graph-based pathway search, and shared constants spanning structure and kinetics. By using the same cohesion-quantum logic across multiple modules, the disclosed system can produce deterministic folding paths, structure reconstructions, kinetic profiles, co-transcriptional timing interactions, accelerator-compatible processing flows, and reproducible benchmark records within one coherent computational framework. The figures and corresponding numerals described herein illustrate representative embodiments and do not limit the disclosed subject matter to only the illustrated implementations.

In state-selection policy embodiments, the disclosed engine can use an ordered decision policy to choose among multiple candidate state representations before graph solving begins. In some embodiments, a first policy stage selects a state library based on molecule type, such as protein, RNA, DNA, enzyme, membrane protein, or binding-site evaluation. In some embodiments, a second policy stage selects a discretization family, such as a six-region backbone discretization, an expanded backbone discretization, a rotamer-augmented discretization, or a reduced kinetics-only discretization. In some embodiments, a third policy stage selects a cost regime, such as destination-state cost, differential cost, tunneling-derived cost, or a hybrid cost. In some embodiments, a fourth policy stage selects a solver regime, such as Dijkstra, A-star, or layered dynamic programming. In some embodiments, each policy stage is selected from a finite predefined set so that the resulting run remains objectively bounded and reproducible.

In calibration handling, the disclosed engine distinguishes a fixed disclosed cohesion quantum from implementation-level numeric representation details. In some embodiments, E_coh_RS is treated as the Recognition-Physics native expression and E_coh_eV is treated as the fixed calibrated value used for numerical simulation. In some embodiments, the disclosed engine stores both values in a constants package and prevents run-time tuning of those constants absent an explicit authorized configuration change. In some embodiments, a calibration record stores the exact numeric representation used in a given implementation, including decimal precision, storage type, and any fixed-point encoding used on specialized hardware. In some embodiments, this prevents a later reviewer from confusing a numerical encoding choice with an underlying change to the disclosed ladder architecture.

In constants management embodiments, the disclosed engine can implement a constants registry that stores the cohesion quantum, temperature defaults, polymerase-prefactor values, ladder-index mapping tables, graph-policy identifiers, and benchmark identifiers as versioned assets. In some embodiments, each simulation run references a specific constants registry version. In some embodiments, a run is rejected if a required constants entry is missing or inconsistent. In some embodiments, a result artifact stores the constants registry version so that later replay and audit can be performed without reconstructing assumptions from source code alone.

In graph-admissibility policy embodiments, the disclosed engine can apply ordered admissibility rules before emitting an edge between candidate states. In some embodiments, the ordered rules include: 1. verify sequence adjacency or other allowed state-order relationship; 2. verify that the candidate transition does not violate a configured state-span threshold, such as an absolute ladder-index change greater than a predefined bound; 3. verify that the candidate transition satisfies any applicable residue-class or nucleotide-class compatibility rule; 4. verify that the candidate transition does not violate any configured steric or exclusion condition; and 5. emit the edge only if the prior checks succeed. In some embodiments, each failed check is logged by category so that graph sparsity and solver behavior can be explained after the run.

In source-state and target-state definition embodiments, the disclosed engine can support multiple objective definitions while preserving determinism. In some embodiments, a source state is defined as a highest-supported ladder state at a first sequence position, an unfolded-state representative selected from a finite set, or a configured boundary-state tuple. In some embodiments, a target state is defined as a ladder-zero state at a final sequence position, a lowest-cost admissible terminal state, or a terminal state satisfying an independently configured native-state rule. In some embodiments, where more than one source state or target state is permitted, the solver can be executed in multi-source or multi-target mode while still retaining an objective minimum-cost outcome.

In pathway-ranking embodiments, the disclosed engine can generate more than a single winning path. In some embodiments, the engine stores the top K lowest-cost paths, where K is a configured positive integer. In some embodiments, the engine stores all tied lowest-cost paths and then ranks non-tied alternatives by total cost, state-diversity score, or another predefined secondary metric. In some embodiments, this ranking supports downstream ensemble calculations, pathway comparison, trapped-state analysis, or experimental candidate selection. In some embodiments, the ranked-path output is preserved as a machine-actionable artifact distinct from any human-readable report.

In trapped-state analysis embodiments, the disclosed engine can identify locations or states associated with elevated cumulative path cost, repeated local minima, repeated tie conditions, or abrupt cost increases. In some embodiments, a trapped-state indicator is assigned to a residue position, nucleotide position, or local state block when the local path cost exceeds a threshold selected from a finite predefined policy set. In some embodiments, trapped-state indicators are used to label potential folding bottlenecks, pause bottlenecks, aggregation-prone regions, or intervention points. In some embodiments, those indicators are output separately from the core path so that detection is distinguishable from any downstream recommendation or action.

In structure-confidence embodiments, the disclosed engine can compute one or more confidence indicators from internal consistency measures rather than from an external learned confidence model. In some embodiments, a confidence indicator is derived from the gap between the best path cost and the next-best path cost. In some embodiments, a confidence indicator is derived from the fraction of total Boltzmann weight captured by the selected path or by the selected path family. In some embodiments, a confidence indicator is derived from reconstruction success, state consistency, graph connectivity density, or repeated reproducibility of the same path under identical settings. In some embodiments, such indicators are reported as explanatory metadata and not as independent physical quantities.

In local-versus-distributed execution embodiments, the disclosed engine can be deployed on a single workstation, a laboratory server, a cloud cluster, an edge device, or a hybrid environment. In some embodiments, local execution retains all graph construction, solving, and output generation within one machine boundary. In some embodiments, distributed execution partitions preprocessing, graph generation, path solving, and reporting across multiple nodes. In some embodiments, distributed execution is coordinated by a deterministic scheduler so that work partitioning does not change the substantive output of a run given the same input and configuration. In some embodiments, any nondeterministic scheduling layer is insulated from substantive computation by requiring deterministic merge and reduction operations.

In batch-processing embodiments, the disclosed engine accepts a batch of sequences and processes them under a shared constants package. In some embodiments, each sequence in a batch receives an independent run identifier and output package. In some embodiments, the engine can share lookup tables, graph templates, or hardware kernels across the batch to improve throughput. In some embodiments, batched outputs are accompanied by per-item provenance so that one item's results are not confused with another item's results even when both are produced in the same execution job.

In offline and online embodiments, the disclosed engine supports both precomputed and live-computed operation. In some embodiments, an offline embodiment precomputes ladder tables, admissibility tables, benchmark comparisons, or reference graph fragments before a user request arrives. In some embodiments, an online embodiment computes all requested outputs after request receipt. In some embodiments, a hybrid embodiment uses cached structural assets while still performing sequence-specific path solving or kinetics generation at request time. In some embodiments, outputs clearly identify whether a component was computed live or retrieved from cache.

In caching embodiments, the disclosed engine can cache one or more of normalized sequence representations, ladder-index arrays, graph fragments, path-solver intermediate states, structure reconstructions, benchmark comparisons, or proof-check results. In some embodiments, cache keys include at least the input identity and the relevant ruleset identity. In some embodiments, a cache hit is valid only if the current constants package, mapping tables, solver policy, and precision policy match the cached record. In some embodiments, the engine rejects stale cache entries rather than silently mixing outputs produced under different rulesets.

In validation embodiments, the disclosed engine can perform internal validation before returning a result. In some embodiments, internal validation includes: 1. confirming that the input was normalized successfully; 2. confirming that required constants are present; 3. confirming that graph construction produced at least one valid source-to-target path where such a path is required; 4. confirming that edge costs satisfy configured solver requirements; 5. confirming that reconstruction succeeded where a structural output is requested; and 6. confirming that output serialization completed successfully. In some embodiments, each validation result is stored as structured metadata and returned as part of a run report.

In benchmark-comparison embodiments, the disclosed engine distinguishes internal validation from external benchmarking. In some embodiments, internal validation answers whether the run was computationally well-formed. In some embodiments, external benchmarking answers how the output compares with a reference dataset. In some embodiments, a run can pass internal validation without matching a reference structure or reference histogram. In some embodiments, a run can be benchmarked only when a reference dataset is present and appropriately associated. In some embodiments, these distinctions reduce ambiguity in prosecution and technical review by showing that runtime success, reference agreement, and claim scope are different questions.

In error-handling embodiments, the disclosed engine can emit explicit machine-readable error states rather than returning partial or ambiguous results. In some embodiments, error classes include unsupported input symbols, missing constants, incompatible policy combinations, empty graph generation, no-path conditions, numeric overflow, numeric underflow, reconstruction failure, unavailable accelerator resources, proof-check failure, and serialization failure. In some embodiments, each error class is associated with a deterministic code and a human-readable explanation. In some embodiments, recoverable errors trigger a defined fallback policy, while nonrecoverable errors terminate the run with an explicit failure artifact.

In fallback-policy embodiments, the disclosed engine can apply one or more predefined fallback steps when a primary run configuration fails. In some embodiments, the fallback steps are strictly ordered, such as: 1. retry with a widened state library; 2. retry with an alternative solver from a finite approved set; 3. retry with a relaxed but predefined admissibility policy; and 4. if still unsuccessful, emit a no-result condition. In some embodiments, the engine records whether the final output arose from the primary policy path or from a fallback path. In some embodiments, this distinction is important because it separates core behavior from recovery behavior and preserves transparency for later review.

In manufacturing and deployment embodiments, the disclosed engine can be packaged as a workstation application, a server-resident service, a containerized compute image, a hardware-accelerated appliance, a cloud-native microservice, or a hybrid deployment with local preprocessing and remote solving. In some embodiments, a packaged deployment includes frozen constants, frozen mapping tables, validated kernels, and an associated proof-artifact bundle. In some embodiments, this packaging improves controlled deployment for regulated or production environments by reducing the risk of silent configuration drift.

In continuous-integration embodiments, the disclosed engine can be incorporated into a software-release pipeline in which code changes trigger one or more of proof checks, benchmark replay, deterministic output comparison, regression testing, and packaging validation. In some embodiments, a release is blocked if a required proof artifact fails, if a benchmark deviates outside an approved range, or if a deterministic replay test produces an inconsistent result under unchanged inputs and constants. In some embodiments, this CI/CD use case provides additional support that the disclosed system is an engineering-capable architecture rather than a purely theoretical proposal.

In audit embodiments, the disclosed engine can generate a run ledger that records input identity, normalization actions, constants identity, mapping-table identity, graph-policy identity, solver identity, precision policy, fallback-policy use, output identity, and benchmark association. In some embodiments, the run ledger is cryptographically hashed or otherwise protected against undetected alteration. In some embodiments, an audit artifact is distinct from the substantive simulation output so that provenance is distinguishable from the biomolecular prediction itself. In some embodiments, such separation improves evidentiary clarity for internal review, regulatory review, and later patent enforcement analysis.

In regulatory and controlled-use embodiments, the disclosed engine can be configured to require approval of constants packages or rule tables before use in production. In some embodiments, only signed or approved rulesets may be loaded by a production instance. In some embodiments, experimental or developmental rulesets are segregated from approved rulesets and clearly labeled in outputs. In some embodiments, this prevents a user from confusing a developmental run with a controlled production run.

In data-representation embodiments, the disclosed engine is not limited to any one internal representation. In some embodiments, states are represented as integer tuples. In some embodiments, states are represented as packed bit fields. In some embodiments, graphs are represented as sparse matrices, adjacency lists, compressed edge arrays, layered tables, or generator functions. In some embodiments, output structures are represented as coordinate arrays, residue-wise records, path objects, or serialized files. In some embodiments, these representation choices do not alter the disclosed invention so long as the fixed cohesion quantum, integer ladder assignments, and explicit state-transition computation remain operative.

In hardware embodiments, the disclosed engine can be realized using one or more processors, one or more memories, configurable logic, lookup tables, arithmetic pipelines, device buffers, and communication interfaces arranged to implement the described functions. In some embodiments, a “module,” “engine,” “pipeline,” or “interface” corresponds to executable instructions carried by a memory and executed by a processor. In some embodiments, such a component corresponds to dedicated hardware or configurable hardware. In some embodiments, mixed implementations are used, such as software-controlled dispatch with hardware-executed kernels.

In device-interface embodiments, outputs can be delivered to a local display, remote client, file store, laboratory information system, workflow scheduler, or downstream analytics engine. In some embodiments, a human-readable display includes structure images, histograms, pathway summaries, or ranked candidate tables. In some embodiments, a machine-actionable output includes coordinate data, state-trace data, graph-trace data, ladder-index arrays, dwell arrays, or API payloads. In some embodiments, the system intentionally separates presentation outputs from substantive computational outputs so that a downstream consumer can act on the machine representation without relying on any specific visualization layer.

In terminology boundaries, the disclosed specification uses stable distinctions to reduce ambiguity. In some embodiments, a “state” is not necessarily a final conformation. In some embodiments, a “pathway” is not necessarily unique. In some embodiments, a “prediction” is not necessarily a guarantee of physical truth for all experimental contexts. In some embodiments, a “benchmark” is not necessarily a universal performance floor. In some embodiments, a “module” is not necessarily a separate physical box and can be a logical or functional partition within a larger implementation. In some embodiments, these clarifications support claim interpretation without narrowing the invention to a single packaging style.

In alternative ladder-mapping embodiments, the specific mapping examples described herein are illustrative and not limiting unless expressly recited otherwise. In some embodiments, a protein embodiment uses six backbone regions. In some embodiments, a protein embodiment uses a finer backbone discretization. In some embodiments, an RNA embodiment uses a base-identity mapping as described above. In some embodiments, an RNA embodiment uses a base-step or context-window mapping from a finite predefined family. In some embodiments, each such mapping remains within the disclosed invention when it preserves the use of integer ladder indices tied to the fixed cohesion quantum.

In alternative solver embodiments, the disclosed invention is not limited to a single graph solver unless later claims expressly say so. In some embodiments, the graph is solved using Dijkstra's algorithm because edge costs are nonnegative. In some embodiments, the graph is solved using A-star search where an admissible heuristic is available. In some embodiments, the graph is solved using layered dynamic programming where the graph structure permits it. In some embodiments, equivalent objective solvers operating on the disclosed graph and cost architecture are also within the scope of the invention.

In alternative output embodiments, one implementation can return only a final structure, another can return a final structure and a minimum-cost path, another can return a ranked path family, and another can return structure, kinetics, benchmark, and proof artifacts together. In some embodiments, the same core engine supports all such variants through output-mode selection rather than through substantive architectural change. In some embodiments, this flexibility provides fallback support for later claim drafting while maintaining consistent terminology across the specification.

In enablement support, the disclosed specification provides objective mechanisms for state assignment, graph generation, transition-cost computation, pathway solving, kinetic calculation, timing coupling, hardware implementation, validation, benchmarking, and fallback behavior. In some embodiments, these mechanisms are sufficient to show possession of the invention and to guide implementation without requiring an examiner or reader to invent the core logic independently. In some embodiments, later claims can draw from the disclosed alternatives, decision rules, data structures, outputs, deployment modes, and safeguards without outrunning the present disclosure.

In written-description support, the disclosed specification repeatedly links the same core architecture to multiple embodiments, including protein folding, RNA kinetics, co-transcriptional folding, compound ranking, cloud deployment, GPU execution, FPGA execution, and formal verification. In some embodiments, that repeated use of stable terms and repeated explanation of the same core mechanics demonstrates that the inventors possessed not merely a desired result but a coherent and reusable technical architecture. In some embodiments, the alternatives described herein provide fallback support for narrower and broader claims without introducing new matter.

In concluding scope language, embodiments described herein are illustrative and non-limiting. Features described in connection with one embodiment can be combined with features described in connection with another embodiment unless the combination is logically incompatible or expressly excluded. No figure count beyond FIGS. 1-12 is required for practicing the invention as disclosed herein. The reference numerals described in this specification are provided to facilitate understanding and are not intended to require any particular physical scale, layout, drawing style, or implementation packaging unless later claims expressly require otherwise. The invention encompasses the full range of systems, methods, devices, services, and machine-readable implementations falling within the scope of the appended claims, as supported by the present disclosure.

1. A computer-implemented method for biomolecular simulation, comprising: receiving biomolecular input data identifying a biomolecular sequence; assigning, by one or more processors, integer ladder indices to a plurality of discrete biomolecular states associated with the biomolecular sequence; computing, from a predetermined cohesion-quantum value fixed for a selected ruleset comprising a finite predefined combination of ladder-index mapping policy, admissibility policy, and solver policy before pathway solving begins, respective state energies for the discrete biomolecular states, each state energy being an integer multiple of the predetermined cohesion-quantum value; constructing a layered discrete state graph having nodes corresponding to candidate states for successive sequence positions and edges corresponding only to transitions satisfying one or more predefined admissibility rules selected from sequence adjacency, ladder-index-span threshold, residue-class compatibility, nucleotide-class compatibility, and steric or exclusion checks; computing nonnegative transition costs for the edges as at least one of a destination-state ladder-energy cost, a nonnegative ladder-index-difference cost, or an additive tunneling-derived cost, wherein candidate transitions producing a negative value are rejected before solver execution; determining, by a shortest-path solver, an ordered state pathway through the layered discrete state graph; and generating an output from the ordered state pathway, the output comprising a predicted biomolecular structure and a pathway record for a protein-folding embodiment.

2. The method of claim 1, wherein assigning the integer ladder indices comprises assigning a respective ladder index to each of a plurality of discretized backbone torsion states of a protein using a predefined mapping table that maps six discretized backbone torsion-state bins respectively to ladder indices 0, 1, 2, 3, 4, and 5.

3. The method of claim 2, wherein constructing the discrete state graph comprises constructing a layered torsion-state graph in which nodes correspond to candidate states for successive residue positions and edges correspond to permitted transitions between candidate states of adjacent residue positions that satisfy predefined admissibility rules comprising sequence adjacency, ladder-index-span threshold, residue-class compatibility, and steric exclusion checks.

4. The method of claim 3, wherein determining the pathway comprises applying a shortest-path solver to the layered torsion-state graph using the nonnegative transition costs and a deterministic tie-resolution rule comprising lower cumulative high-index count, lower lexicographic state identifier, lower residue-wise variance, or earlier discovery order under a fixed queue policy.

5. The method of claim 4, wherein the shortest-path solver comprises Dijkstra's algorithm operating on nonnegative edge costs and emitting predecessor records, final costs, and a tie-resolution record for path reconstruction.

6. The method of claim 1, wherein computing the nonnegative transition costs comprises computing an additive transition cost as a negative logarithm of a tunneling weight associated with a permitted transition between adjacent ladder levels and combining the additive transition cost with a destination-state ladder-energy cost or a nonnegative ladder-index-difference cost.

7. The method of claim 6, wherein the tunneling weight is based on an exponential function of the fixed cohesion quantum and a characteristic frequency term, and wherein in locked embodiments the additive transition cost is a fixed constant equal to 1 per adjacent ladder step.

8. The method of claim 1, wherein generating the output comprises reconstructing a three-dimensional biomolecular structure from a sequence of discrete states along the pathway using internal-coordinate reconstruction with bond lengths, bond angles, torsion angles, and chirality constraints, and outputting a pathway record associated with the reconstructed three-dimensional biomolecular structure.

9. The method of claim 1, further comprising generating a machine-actionable artifact comprising at least one of a predecessor table, a path-cost decomposition, a ladder-index sequence, a coordinate file, a reconstruction-status record, a node trace, an edge trace, or a provenance record configured for deterministic replay.

10. A computer-implemented method for nucleic-acid kinetics, comprising: receiving nucleic-acid input data identifying a nucleic-acid sequence; assigning nucleotide-position ladder indices according to a predefined base-identity mapping in which adenine maps to 0, thymine or uracil maps to 1, guanine maps to 2, and cytosine maps to 3; computing position-dependent translocation rates from the assigned nucleotide-position ladder indices and a predetermined cohesion-quantum value fixed for a selected ruleset comprising a finite predefined combination of base-identity mapping policy, polymerase-prefactor policy, and normalization policy before rate computation begins, wherein the position-dependent translocation rates are computed using an exponential rate law; computing dwell times from the position-dependent translocation rates; normalizing the dwell times to produce a pause-density output; and generating an output comprising a kinetic profile or the pause-density output.

11. The method of claim 10, wherein the predefined base-identity mapping is stored as a fixed lookup table, wherein a polymerase-specific prefactor is retrieved from a registry associated with a selected polymerase species or polymerase class and is not refit per sequence during the run, and wherein the output further comprises a dwell-time array, a run identifier, and a machine-readable trace indicating how each rate was computed from underlying ladder assignments.

12. The method of claim 11, further comprising coupling the pause-density output or dwell times to a co-transcriptional folding simulation by exposing a timing window to a folding module for a nascent transcript, wherein the timing window limits state eligibility in the folding module according to currently available transcript length and accumulated dwell time before a next transcription increment occurs.

13. A biomolecular simulation system, comprising: one or more processors; and a memory storing instructions that, when executed by the one or more processors, cause the system to: receive biomolecular input data; assign integer ladder indices to discrete biomolecular states associated with the biomolecular input data; compute state energies as integer multiples of a predetermined cohesion-quantum value fixed for a selected ruleset comprising a finite predefined combination of ladder-index mapping policy, admissibility policy, and solver policy before pathway solving begins; construct a layered discrete state graph having nodes corresponding to candidate states for successive sequence positions and edges corresponding only to transitions satisfying one or more predefined admissibility rules selected from sequence adjacency, ladder-index-span threshold, residue-class compatibility, nucleotide-class compatibility, and steric or exclusion checks; compute nonnegative transition costs for the edges as at least one of a destination-state ladder-energy cost, a nonnegative ladder-index-difference cost, or an additive tunneling-derived cost, wherein candidate transitions producing a negative value are rejected before solver execution; determine, by a shortest-path solver, an ordered state pathway through the layered discrete state graph; and generate an output from the ordered state pathway, the output comprising a predicted biomolecular structure and a pathway record for a protein-folding embodiment.

14. The system of claim 13, wherein the instructions further cause the system to assign the integer ladder indices using a predefined mapping table stored in the memory, the predefined mapping table mapping six discretized backbone torsion-state bins respectively to ladder indices 0, 1, 2, 3, 4, and 5.

15. The system of claim 13, wherein the instructions further cause the system to compute the nonnegative transition costs from destination-state ladder energy, nonnegative ladder-index difference, tunneling-derived cost, or a combination thereof, wherein a hybrid edge cost is formed as a sum of destination-state cost and tunneling-derived cost and candidate transitions producing a negative value are rejected before solver execution, and wherein in locked embodiments the tunneling-derived cost is a fixed constant equal to 1 per adjacent ladder step.

16. The system of claim 13, further comprising a graphics processing unit configured to execute at least one of ladder assignment, graph construction, pathway determination for layered sparse graphs, ensemble averaging, or kinetic-profile computation, wherein integer ladder indices are stored in integer arrays, ladder energies are stored in FP32 arrays, path accumulators or exponential computations are performed in FP32 or FP64 according to a numeric-stability policy, and FP16 intermediate steps, when used, preserve a higher-precision accumulation path for cost totals or exponentials.

17. The system of claim 13, further comprising a field-programmable gate array configured to implement at least one of a ladder lookup table, a tunneling cost engine, a shortest-path solver pipeline, or an RNA kinetics pipeline, wherein in locked embodiments the field-programmable gate array implements a fixed per-step tunneling cost equal to 1, wherein the ladder lookup table stores ladder energies for a supported range of integer ladder values, and wherein the shortest-path solver pipeline retains predecessor traces in on-chip memory or staged off-chip memory for later path reconstruction.

18. The system of claim 13, wherein the instructions further cause the system to generate a traceability record identifying a constants package version, a mapping-rule package version, a solver-policy identifier, a precision-policy identifier, a run identifier, a hardware identifier, a ruleset identifier, and a benchmark identifier, and to generate a provenance record configured for deterministic replay under the same input, constants, mapping tables, solver policy, and precision policy.

19. The system of claim 13, wherein the instructions further cause the system to perform a validation process comprising confirming that required constants are present, verifying successful normalization of the biomolecular input data, verifying graph solvability for a requested pathway output, verifying that edge costs satisfy configured solver requirements, verifying that reconstruction succeeded where a structural output is requested, verifying that output serialization completed successfully, distinguishing a validation result from a benchmark-comparison result, and generating a validation artifact distinct from a benchmark-comparison artifact.

20. A non-transitory computer-readable medium storing instructions that, when executed by one or more processors, cause the one or more processors to: receive biomolecular input data identifying a biomolecular sequence; assign integer ladder indices to a plurality of discrete biomolecular states associated with the biomolecular sequence; compute respective state energies for the discrete biomolecular states as integer multiples of a predetermined cohesion-quantum value fixed for a selected ruleset before pathway solving begins; construct a layered discrete state graph; compute nonnegative transition costs for edges of the layered discrete state graph; determine an ordered state pathway through the layered discrete state graph; generate a predicted biomolecular structure and a pathway record from the ordered state pathway; generate a machine-actionable artifact comprising at least one of a predecessor table, a path-cost decomposition, a ladder-index sequence, a coordinate file, a reconstruction-status record, a node trace, an edge trace, or a provenance record configured for deterministic replay; emit a no-path condition when no valid path exists from a configured source to a configured target; record whether a final output arose from a primary policy path or from a fallback path; emit a deterministic error code associated with the no-path condition; and distinguish recoverable and nonrecoverable error classes.
